(19) United States
(12) Patent Application Publication
(10) Pub. No.: US 2006/0167087 A1
(43) Pub. Date: Jul. 27, 2006

(54) Substituted aniline derivatives

(75) Inventors: Daniel R. Greve, Stenlose (DK); Mario Rottlander, Greve (DK); William P Watson, Vanlose (DK)

Correspondence Address: DARBY & DARBY P.C., P. O. BOX 5257, NEW YORK, NY, 10150-5257, US

(73) Assignee: H. Lundbeck A/S, Valby-Copenhagen (DK)

(21) Appl. No.: 10/549,345

(22) PCT Filed: Mar. 12, 2004
(86) PCT No.: PCT/DK04/00162

§ 371 Date: Jan. 31, 2006

Related U.S. Application Data
(60) Provisional application No. 60/454,580, filed on Mar. 14, 2003.

(30) Foreign Application Priority Data
Mar. 14, 2003 (DK) PA200300392

(51) Int. Cl. A61K 31/325; A61K 31/277

(52) U.S. Cl. 514/486; 560/28; 514/521; 558/410

(57) ABSTRACT

The present invention relates to aniline derivatives of the general formula I or pharmaceutically acceptable salts thereof and their use.

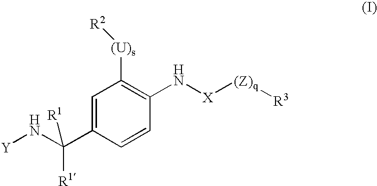

### FIELD OF THE INVENTION

The present invention relates to novel substituted aniline derivatives being openers of the KCNQ family potassium ion channels. The compounds are useful for the prevention, treatment and inhibition of disorders and diseases being responsive to opening of the KCNQ family potassium ion channels, one such disease is epilepsy.

### BACKGROUND OF THE INVENTION

Ion channels are cellular proteins that regulate the flow of ions, including potassium, calcium, chloride and sodium into and out of cells. Such channels are present in all animal and human cells and affect a variety of processes including neuronal transmission, muscle contraction, and cellular secretion.

Humans have over 70 potassium channel subunits (Jentsch Nature Reviews Neuroscience 2000, 1, 21-30) with a great diversity with regard to both structure and function. Neuronal potassium channels, which are found in the brain, are primarily responsible for maintaining a negative resting membrane potential, as well as controlling membrane repolarisation following an action potential.

One subset of potassium channel genes is the KCNQ family. Mutations in four out of five KCNQ genes have been shown to underlie diseases including cardiac arrhythmias, deafness and epilepsy (Jentsch Nature Reviews Neuroscience 2000, 1, 21-30).

The KCNQ4 gene is thought to encode the molecular correlate of potassium channels found in outer hair cells of the cochlea and in Type I hair cells of the vestibular apparatus, in which mutations lead to a form of inherited deafness.

KCNQ1 (KvLQT1) is co-assembled with the product of the KCNE1 (minimal K(+)-channel protein) gene in the heart to form a cardiac-delayed rectifier-like K(+) current. Mutations in this channel can cause one form of inherited long QT syndrome type 1 (LQT1), as well as being associated with a form of deafness (Robbins Pharmacol Ther 2001, 90, 1-19).

The genes KCNQ2 and KCNQ3 were discovered in 1988 and appear to be mutated in an inherited form of epilepsy known as benign familial neonatal convulsions (Rogawski Trends in Neurosciences 2000, 23, 393-398). The proteins encoded by the KCNQ2 and KCNQ3 genes are localised in the pyramidal neurons of the human cortex and hippocampus, regions of the brain associated with seizure generation and propagation (Cooper et al. Proceedings National Academy of Science USA 2000, 97, 4914-4919).

KCNQ2 and KCNQ3 are two potassium channel subunits that form “M-currents” when expressed in vitro. The M-current is a non-inactivating potassium current found in many neuronal cell types. In each cell type, it is dominant in controlling membrane excitability by being the only sustained current in the range of action potential initiation (Marrion Annual Review Physiology 1997, 59, 483-504). Modulation of the M-current has dramatic effects on neuronal excitability, for example activation of the current will reduce neuronal excitability. Openers of these KCNQ channels or activators of the M-current, will reduce excessive neuronal activity and may thus be of use in the treatment, prevention or inhibition of seizures and other diseases and disorders characterised by excessive neuronal activity, such as neuronal hyperexcitability including convulsive disorders, epilepsy and neuropathic pain.

Retigabine (D-23129; N-(2-amino-4-(4-fluorobenzylamino)-phenyl)carbamic acid ethyl ester) and analogues thereof are disclosed in EP554543. Retigabine is an anti-convulsive compound with a broad spectrum and potent anticonvulsant properties, both in vitro and in vivo. It is active after oral and intraperitoneal administration in rats and mice in a range of anticonvulsant tests including: electrically induced seizures, seizures induced chemically by pentylenetetrazole, picrotoxin and N-methyl-D-aspartate (NMDA) and in a genetic animal model, the DBA/2 mouse (Rostock et al. Epilepsy Research 1996, 23, 211-223). In addition, retigabine is active in the amygdala kindling model of complex partial seizures, further indicating that this compound has potential for anti-convulsive therapy. In clinical trials, retigabine has recently shown effectiveness in reducing the incidence of seizures in epileptic patients (Bialer et al. Epilepsy Research 2002, 51, 31-71).

Retigabine has been shown to activate a K(+) current in neuronal cells and the pharmacology of this induced current displays concordance with the published pharmacology of the M-channel, which recently was correlated to the KCNQ2/3 K(+) channel heteromultimer. This suggests that activation of KCNQ2/3 channels may be responsible for some of the anticonvulsant activity of this agent (Wickenden et al. Molecular Pharmacology 2000, 58, 591-600)—and that other agents working by the same mechanism may have similar uses.

KCNQ 2 and 3 channels have also been reported to be upregulated in models of neuropathic pain (Wickenden et al. Society for Neuroscience Abstracts 2002, 454.7), and potassium channel modulators have been hypothesised to be active in both neuropathic pain and epilepsy (Schroder et al. Neuropharmacology 2001, 40, 888-898).

Retigabine has also been shown to be beneficial in animal models of neuropathic pain (Blackburn-Munro and Jensen European Journal of Pharmacology 2003, 460, 109-116), and we thus suggest that openers of KCNQ channels will be of use in treating pain disorders including neuropathic pain.

The localisation of KCNQ channel mRNA is reported in brain and other central nervous system areas associated with pain (Goldstein et al. Society for Neuroscience Abstracts 2003, 53.8).

In addition to a role in neuropathic pain, the expression of mRNA for KCNQ 2-5 in the trigeminal and dorsal root ganglia and in the trigeminal nucleus caudalis implies that openers of these channels may also affect the sensory processing of migraine pain (Goldstein et al. Society for Neuroscience Abstracts 2003, 53.8).

Recent reports demonstrate that mRNA for KCNQ 3 and 5, in addition to that for KCNQ2, are expressed in astrocytes and glial cells. Thus KCNQ 2, 3 and 5 channels may help modulate synaptic activity in the CNS and contribute to the neuroprotective effects of KCNQ channel openers (Noda et al., Society for Neuroscience Abstracts 2003, 53.9).

Retigabine and other KCNQ modulators may thus exhibit protection against the neurodegenerative aspects of epilepsy, as retigabine has been shown to prevent limbic neurodegeneration and the expression of markers of apoptosis following kainic acid-induced status epilepticus in the rat (Ebert et al. Epilepsia 2002, 43 Suppl 5, 86-95). This may have relevance for preventing the progression of epilepsy in patients, i.e. be anti-epileptogenic. Retigabine has also been shown to delay the progression of hippocampal kindling in the rat, a further model of epilepsy development (Tober et al. European Journal Of Pharmacology 1996, 303, 163-169).

Thus we suggest that these properties of retigabine and other KCNQ modulators may prevent neuronal damage induced by excessive neuronal activation, and may be of use in the treatment of neurodegenerative diseases, and be disease modifying (or antiepileptogenic) in patients with epilepsy.

Given that anticonvulsant compounds such as benzodiazepines and chlormethiazole are used clincially in the treatment of the ethanol withdrawal syndrome and that other anticonvulsant compounds e.g. gabapentin, are very effective in animal models of this syndrome (Watson et al. Neuropharmacology 1997, 36, 1369-1375), we expect that other anticonvulsant compounds such as KCNQ openers will also be effective in this condition.

mRNA for KCNQ 2 and 3 subunits are found in brain regions associated with anxiety and emotional behaviours such as bipolar disorder e.g. hippocampus and amygdala (Saganich et al. Journal of Neuroscience 2001, 21, 4609-4624), and retigabine is reportedly active in some animal models of anxiety-like behaviour (Hartz et al. Journal of Psychopharmacology 2003, 17 suppl 3, A28,B16), and other clinically used anticonvulsant compounds are used in the treatment of bipolar disorder.

WO 200196540 discloses the use of modulators of the M-current formed by expression of KCNQ2 and KCNQ3 genes for insomnia, while WO 2001092526 discloses that modulators of KCNQ5 can be utilized for the treatment of sleep disorders.

WO01/022953 describes the use of retigabine for prophylaxis and treatment of neuropathic pain such as allodynia, hyperalgesic pain, phantom pain, neuropathic pain related to diabetic neuropathy and neuropathic pain related to migraine.

WO02/049628 describes the use of retigabine for the prevention, treatment, inhibition and amelioration of anxiety disorders such as anxiety, generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia and specific phobias.

WO97/15300 describes the use of retigabine for the treatment of neurodegenerative disorders such as Alzheimer's; Huntington's; sclerosis such as multiple sclerosis and amyotrophic lateral sclerosis; Creutzfeld-Jakob disease; Parkinson's disease; AIDS-induced encephalopathy and other infection-related encephalopathies being caused by rubella viruses, herpes viruses, borrelia and by unknown pathogens, trauma-induced neurodegenerations, neuronal hyperexcitation states such as in medicament withdrawal or intoxication, and neurodegenerative disorders of the peripheral nervous system such as polyneuropathies and polyneuritides.

Hence, there is a great desire for novel compounds, which are potent openers of the KCNQ family potassium channels.

Also desired are novel compounds with improved properties relative to known compounds, which are openers of the KCNQ family potassium channels, such as retigabine. Improvement of one or more of the following parameters is desired: half-life, clearance, selectivity, interactions with other medications, bioavailability, potency, formulability, chemical stability, metabolic stability, membrane permeability, solubility and therapeutic index. The improvement of such parameters may lead to improvements such as:
an improved dosing regime by reducing the number of required doses a day,
ease of administration to patients on multiple medications,
reduced side effects,
enlarged therapeutic index,
improved tolerability or
improved compliance.

### SUMMARY OF THE INVENTION

One object of the present invention is to provide novel compounds, which are potent openers of the KCNQ family potassium channels.

The compounds of the invention are substituted aniline derivatives of the general formula I or salts thereof 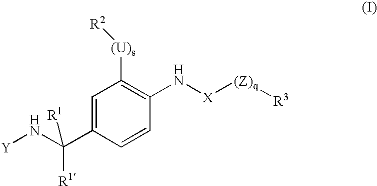
wherein Y, U, X, Z, S, q, R¹, R^{1′}, R² and R³ are as defined below.

The invention further relates to a pharmaceutical composition comprising one or more compounds of formula I and the use thereof.

### DETAILED DESCRIPTION OF THE INVENTION

Accordingly, the present invention relates to substituted aniline derivatives of the general formula I 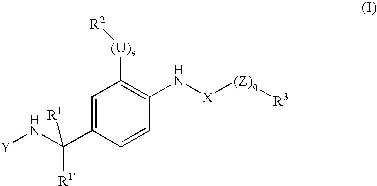
wherein
U is O, S or NR^{2′};
s is 0 or 1;
X is CO or SO₂;
Z is O, S or NR⁴, wherein R⁴ is selected from the group consisting of hydrogen, C₁₋₆-alk(en/yn)yl, C₃₋₈-cycloalk(en)yl, C₃₋₈cycloalk(en)yl-C₁₋₆-alk(en/yn)yl, hydroxy-C₁₋₆-alk(en/yn)yl and hydroxy-C₃₋₈-cycloalk(en)yl;
q is 0 or 1;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl;
R² is selected from the group consisting of hydrogen, halogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl, halo-C₃₋₈-cycloalk(en)yl and cyano; provided that when R² is halogen or cyano, then s is 0;
when s is 1 and U is NR^{2′} then R^{2′} is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; or R² and R^{2′} together form a 5-8 membered saturated or unsaturated ring which optionally contains one further heteroatom;
R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; and
Y represents a group of formulae VI, VII, VIII, IX or XXX: 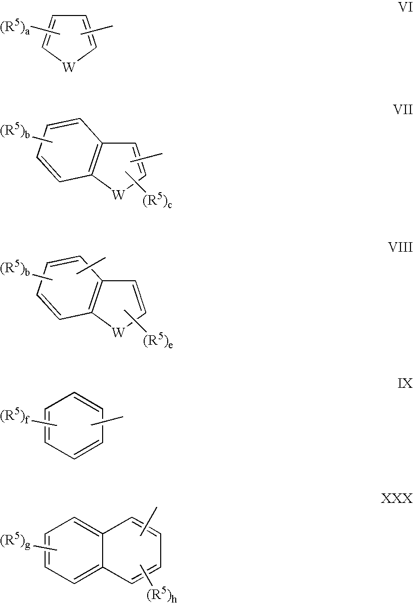
wherein
the line represents a bond attaching the group represented by Y to the nitrogen atom;
W is O or S;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3; and
each R⁵ is independently selected from the group consisting of a C₁₋₆-alk(en/yn)yl, C₃₋₈-cycloalk(en)yl, Ar, C₃₋₈-cycloalk(en)yl-C₁₋₆-alk(en/yn)yl, Ar—C₁₋₆-alk(en/yn)yl, acyl, C₁₋₆-alk(an/en/yn)yloxy, halogen, halo-C₁₋₆-alk(en/yn)yl, —CO—NR⁶R^{6′}, cyano, nitro, —NR⁷R^{7′}, —S—R⁸, —SO₂R⁸ and SO₂OR⁸, or two substituents together form a 5-8 membered saturated or unsaturated ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; with the provisos that when R⁵ is SO₂OR⁸ then R⁸ is not —NR⁹R^{9′} and when R⁵ is SO₂R⁸, then R⁸ is not a hydrogen atom;
or salts thereof.

### DETAILED DESCRIPTION

One embodiment of the invention relates to compounds of the general formula I, wherein R¹ and R^{1′} are independently selected from the group consisting of hydrogen, C₁₋₆-alk(en/yn)yl, C₃₋₈cycloalk(en)yl and C₃₋₈-cycloalk(en)yl-C₁₋₆-alk(en/yn)yl.

In another embodiment, the invention relates to compounds of formula I, wherein R¹ and R^{1′} are independently selected from the group consisting of acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl.

One embodiment of the invention relates to compounds of the general formula I, wherein R¹ is selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl and R^{1′} is selected from the group consisting of acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl.

In yet another embodiment the invention relates to compounds of the general formula I, wherein R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl.

In yet another embodiment, the invention relates to compounds of formula I, wherein one of R¹ and R^{1′} is a hydrogen atom.

In yet another embodiment, the invention relates to compounds of formula I, wherein at least one of R¹ and R^{1′} is a hydrogen atom.

In a preferred embodiment, the invention relates to compounds of formula I, wherein both R¹ and R^{1′} are hydrogen atoms.

In another preferred embodiment, the invention relates to compounds of formula I, wherein R¹ and R^{1′} are independently selected from the group consisting of hydrogen and C₁₋₆-alk(en/yn)yl.

In yet another embodiment, the invention relates to compounds of formula I, wherein 0 is 1.

In a preferred embodiment, the invention relates to compounds of formula I, wherein s is 0.

In yet another embodiment, the invention relates to compounds of the general formula I, wherein R² is selected from the group consisting of Ar, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl.

In yet another embodiment, the invention relates to compounds of the general formula I, wherein R² is selected from the group consisting of hydrogen, halogen, cyano, C₁₋₆-alk(en/yn)yl, C₃₋₈-cycloalk(en)yl, C₃₋₈-cycloalk(en)yl-C₁₋₆-alk(en/yn)yl, Ar—C₁₋₆-alk(en/yn)yl and Ar—C₃₋₈-cycloalk(en)yl; provided that when R² is halogen or cyano, then s is 0.

In yet another embodiment, the invention relates to compounds of the general formula I, wherein R² is selected from the group consisting of hydrogen, halogen, cyano, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl and Ar—C₃₋₈-cycloalk(en)yl; provided that when R² is halogen or cyano, then s is 0.

In yet another embodiment, the invention relates to compounds of the general formula I, wherein R² is selected from the group consisting of hydrogen, halogen, C₁₋₆-alk(en/yn)yl and Ar; provided that when R² is halogen, then s is 0.

In yet another embodiment, the invention relates to compounds of the general formula I, wherein s is 0 and R² is selected from the group consisting of hydrogen, halogen, C₁₋₆-alk(en/yn)yl and Ar.

In yet another embodiment, the invention relates to compounds of formula I, wherein R² is Ar, typically phenyl substituted with halogen or —N(C₁₋₆-alk(en/yn)yl)₂.

In yet another embodiment, the invention relates to compounds of formula I, wherein R² is C₁₋₆-alk(en/yn)yl, typically C₁₋₃-alk(en/yn)yl.

In yet another embodiment, the invention relates to compounds of formula I, wherein R² is C₃₋₈-CYCLOALK(en)yl, typically C₃₋₆-cycloalk(en)yl.

In yet another embodiment, the invention relates to compounds of formula I, wherein R² is C₃₋₈-cycloalk(en)yl-C₁₋₆-alk(en/yn)yl, typically C₃₋₆-cycloalk(en)yl-C₁₋₃-alk(en/yn)yl.

In yet another embodiment, the invention relates to compounds of formula I, wherein R² is Ar—C₁₋₆-alk(en/yn)yl, typically Ar—C₁₋₃-alk(en/yn)yl.

In a preferred embodiment, the invention relates to compounds of the general formula I, wherein R² is Ar—C₃₋₈-cycloalk(en)yl, typically Ar—C₃₋₆-cycloalk(en)yl.

In yet another embodiment, the invention relates to compounds of formula I, wherein s is 0 and R² is different from a hydrogen atom, a halogen atom and C₁₋₆-alkyl.

In yet another embodiment, the invention relates to compounds of formula I, wherein R² is a hydrogen atom.

In yet another embodiment, the invention relates to compounds of formula I, wherein s is 0 and R² is a hydrogen atom.

In yet another embodiment, the invention relates to compounds of formula I, wherein s is 0 and R² is different from a hydrogen atom.

In yet another embodiment, the invention relates to compounds of formula I, wherein s is 1, U is O and R² is different from a hydrogen atom, C₁₋₆-alkyl and acyl.

In yet another embodiment, the invention relates to compounds of the general formula I, wherein s is 1, U is S or NR^{2′} and R² is a hydrogen atom.

In yet another embodiment, the invention relates to compounds of formula I, wherein s is 0 and R² is cyano.

In yet another embodiment, the invention relates to compounds of formula I, wherein s is 0 and R² is a halogen atom such as a fluoro atom or a chloro atom.

In yet another embodiment, the invention relates to compounds of formula I, wherein s is 1 and U is O or S.

In yet another embodiment, the invention relates to compounds of formula I, wherein s is 1 and U is NR^{2′}.

In yet another embodiment, the invention relates to compounds of formula I, wherein s is 1 and U is not NR^{2′}.

In yet another embodiment, the invention relates to compounds of formula I, wherein s is 1, U is NR^{2′} and R² and R^{2′} are independently selected from the group consisting of hydrogen, C₁₋₆-alk(en/yn)yl and Ar.

In yet another embodiment, the invention relates to compounds of the general formula I, wherein s is 1, U is NR^{2′} and R^{2′} is selected from the group consisting of Ar, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl.

In yet another embodiment, the invention relates to compounds of the general formula I, wherein s is 1, U is NR^{2′} and R^{2′} is selected from the group consisting of hydrogen, C₁₋₆-alk(en/yn)yl, C₃₋₈-cycloalk(en)yl, C₃₋₈-cycloalk(en)yl-C₁₋₆alk(en/yn)yl, Ar—C₁₋₆-alk(en/yn)yl and Ar—C₃₋₈-cycloalk(en)yl.

In yet another embodiment, the invention relates to compounds of the general formula I, wherein s is 1, U is NR^{2′} and R² and R^{2′} together form a 5-8 membered saturated or unsaturated ring which optionally contains one further heteroatom.

In yet another embodiment, the invention relates to compounds of formula I, wherein R² and R^{2′} together form pyrrolidin, piperidin, piperazin, morpholin, pyrrol, oxazolidin, thiazolidin or imidazolidin.

In a preferred embodiment, the invention relates to compounds of formula I, wherein s is 1, U is NR^{2′} and none of R² and R^{2′} is a hydrogen atom.

In a preferred embodiment, the invention relates to compounds of formula I, wherein s is 1, U is NR^{2′} and at least one of R² and R^{2′} is a hydrogen atom.

In yet another embodiment, the invention relates to compounds of formula I, wherein s is 1, U is NR^{2′} and R^{2′} is a hydrogen atom.

In a preferred embodiment, the invention relates to compounds of formula I, wherein s is 1, U is NR^{2′} and both R² and R^{2′} are hydrogen atoms.

In yet another embodiment, the invention relates to compounds of formula I, wherein s is 1, U is NR^{2′} and at least one of R² and R^{2′} is different from Ar, Ar—C₁₋₆-alk(en/yn)yl and Ar—C₃₋₈-cycloalk(en)yl.

In yet another embodiment, the invention relates to compounds of formula I, wherein X is SO₂.

In a preferred embodiment, the invention relates to compounds of formula I, wherein X is CO.

In yet another embodiment, the invention relates to compounds of formula I, wherein q is 0.

In a preferred embodiment, the invention relates to compounds of formula I, wherein q is 1.

In yet another embodiment, the invention relates to compounds of formula I, wherein Z is S or NR⁴.

In a preferred embodiment, the invention relates to compounds of formula I, wherein Z is O.

In yet another embodiment, the invention relates to compounds of formula I, wherein R³ is selected from the group consisting of Ar, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl.

In yet another embodiment, the invention relates to compounds of formula I, wherein R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar—C₁₋₆-alk(en/yn)yl and Ar—C₃₋₈-cycloalk(en)yl.

One embodiment of the invention relates to compounds of the general formula I, wherein R³ is Ar; with the proviso that Ar is different from optionally substituted phenyl, optionally substituted condensed phenyl such as naphtyl and optionally substituted thienyl.

One embodiment of the invention relates to compounds of the general formula I, wherein R³ is Ar—C₁₋₆-alk(en/yn)yl; with the proviso that Ar—C₁₋₆-alk(en/yn)yl is different from optionally substituted phenyl-C₁₋₆-alk(en/yn)yl and optionally substituted condensed phenyl-C₁₋₆-alk(en/yn)yl, such as optionally substituted naphtyl-C₁₋₆-alk(en/yn)yl.

Another embodiment of the invention relates to compounds of the general formula I, wherein R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar—C₁₋₆-alk(en/yn)yl, Ar—C₃₋₈-cycloalk(en)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl;

In yet another embodiment, the invention relates to compounds of the general formula I, wherein Y is different from optionally substituted thienyl or phenyl when R³ is Ar—C₁₋₆-alkyl, wherein Ar is optionally substituted naphtyl and C₁₋₆-alk(en/yn)yl is vinylene, 1-propenylene, methylene or ethylene.

In yet another embodiment, the invention relates to compounds of the general formula I, wherein Y is optionally substituted thienyl or phenyl when R³ is different from Ar—C₁₋₆-alkyl, wherein Ar is optionally substituted naphtyl and C₁₋₆-alkyl is vinylene, 1-propenylene, methylene or ethylene.

In yet another embodiment, the invention relates to compounds of formula I, wherein X is CO, q is 0 and R³ is different from C₁₋₄-alkyl, acyl and phenyl optionally being substituted by hydroxyl or C₁₋₄-alkanyloxy.

In yet another embodiment, the invention relates to compounds of formula I, wherein X is CO, q is 0 and R³ is C₁₋₆-alk(en/yn)yl, with the proviso that R³ is different from a methyl group.

In yet another embodiment, the invention relates to compounds of formula I, wherein R³ is C₁₋₆-alk(en/yn)yl, C₃₋₈-cycloalk(en)yl or C₃₋₈-cycloalk(en)yl-C₁₋₆-alk(en/yn)yl.

In a preferred embodiment, the invention relates to compounds of formula I, wherein R³ is C₁₋₆-alk(en/yn)yl.

In yet another embodiment, the invention relates to compounds of formula I, wherein R³ is not a CH₃-group.

In yet another embodiment, the invention relates to compounds of formula I, wherein R³ is a CH₃-group.

In yet another embodiment, the invention relates to compounds of formula I, wherein R³ is ethyl.

In yet another embodiment, the invention relates to compounds of formula I, wherein R³ is isopropyl.

In yet another embodiment, the invention relates to compounds of formula I, wherein R³ is isopropylmethyl.

In yet another embodiment, the invention relates to compounds of formula I, wherein R³ is tert-butylmethyl.

In yet another embodiment, the invention relates to compounds of formula I, wherein R³ is Ar—C₁₋₆-alk(en/yn)yl.

In yet another embodiment, the invention relates to compounds of formula I, wherein R³ is Ar-methyl.

In yet another embodiment, the invention relates to compounds of formula I, wherein Y represents a group of formulae IX or XXX.

In yet another embodiment, the invention relates to compounds of formula I, wherein each R⁵ is independently selected from the group consisting of Ar—C₁₋₆-alk(en/yn)yl, acyl, —CO—NR⁶R^{6′}, cyano, nitro, —NR⁷R^{7′}, —S—R⁸, —SO₂R⁸ and SO₂OR⁸.

In yet another embodiment, the invention relates to compounds of formula I, wherein each R⁵ is independently selected from the group consisting of C₁₋₆-alk(en/yn)yl, C₃₋₈-cycloalk(en)yl, C₃₋₈-cycloalk(en)yl-C₁₋₆-alk(en/yn)yl, Ar, halogen, halo-C₁₋₆-alk(en/yn)yl and C₁₋₆-alk(an/en/yn)yloxy; or two R⁵ together form a 5-8 membered saturated or unsaturated ring which optionally contains one or two heteroatoms.

In a preferred embodiment, the invention relates to compounds of formula I, wherein each R⁵ is independently selected from the group consisting of C₁₋₆-alk(en/yn)yl, C₃₋₈-cycloalk(en)yl, Ar, halogen, halo-C₁₋₆-alk(en/yn)yl and C₁₋₆-alk(an/en/yn)yloxy; or two R⁵ together form a 5-8 membered saturated or unsaturated ring which optionally contains one or two heteroatoms.

In another preferred embodiment, the invention relates to compounds of formula I, wherein each R⁵ is independently selected from the group consisting of C₁₋₆-alk(en/yn)yl, C₃₋₈-cycloalk(en)yl, Ar, halogen, cyano, halo-C₁₋₆-alk(en/yn)yl and C₁₋₆-alk(an/en/yn)yloxy; or two adjacent substituents together form a 5-8 membered saturated or unsaturated ring which optionally contains one or two heteroatoms.

In yet another embodiment, the invention relates to compounds of formula I, wherein each R⁵ is independently selected from the group consisting of C₁₋₆-alk(en/yn)yl, C₃₋₈-cycloalk(en)yl, Ar, halogen, halo-C₁₋₆-alk(en/yn)yl and C₁₋₆-alk(an/en/yn)yloxy.

In yet another embodiment, the invention relates to compounds of formula I, wherein each R⁵ is independently selected from the group consisting of C₁₋₆-alk(en/yn)yl, C₃₋₈-cycloalk(en)yl, Ar, halogen, cyano, halo-C₁₋₆-alk(en/yn)yl and C₁₋₆-alk(an/en/yn)yloxy.

In yet another embodiment, the invention relates to compounds of formula I, wherein two adjacent R⁵ together form a 5-8 membered saturated or unsaturated ring which optionally contains one or two heteroatoms.

In yet another embodiment, the invention relates to compounds of formula I, wherein two adjacent R⁵ together form a 5-8 membered saturated or unsaturated carbocyclic ring.

In yet another embodiment, the invention relates to compounds of formula I, wherein two adjacent R⁵ together form —(CH₂)_{n*}—CH₂—, —CH═CH—(CH₂)_{m*}—, —CH₂—CH═CH—(CH₂)_{p*}—, —(CH₂)_{n*}—O—, —O—(CH₂)_{m*}—O—, —CH₂—O—(CH₂)_{p*}—O—, —CH₂—O—CH₂—O—CH₂—, —(CH₂)_{n*}—S—, —S—(CH₂)_{m*}—S—, —CH₂—S—(CH₂)_{*}—S—, —CH₂—S—CH₂—S—CH₂—, —(CH₂)_{n*}—NH—, —NH—(CH₂)_{m*}—NH—, —CH₂—NH—(CH₂)_{p*}—NH—, —CH═CH—NH—, —O—(CH₂)_{m*}—NH—, —CH₂—O—(CH₂)_{p*}—NH— or —O—(CH₂)_{p*}—NH—CH₂—, —S—(CH₂)_{m*}—NH—, —N═CH—NH—, —N═CH—O— or —N═CH—S—, wherein m* is 1, 2 or 3, n* is 2, 3 or 4 and p* is 1 or 2.

In yet another embodiment, the invention relates to compounds of formula I, wherein two adjacent R⁵ together form —(CH₂)_{n*}—CH₂—, —CH═CH—(CH₂)_{m*}—, —CH₂—CH═CH—(CH₂)_{p*}, wherein m* is 1, 2 or 3, n* is 2, 3 or 4 and p* is 1 or 2.

In yet another embodiment, the invention relates to compounds of formula I, wherein two adjacent R⁵ together form —(CH₂)_{n*}—CH₂— wherein n* is 2, 3 or 4.

In yet another embodiment, the invention relates to compounds of formula I, wherein two adjacent R⁵ together form —(CH₂)₃—

In yet another embodiment, the invention relates to compounds of formula I, wherein one R⁵ is a halogen atom, typically a fluoro or chloro atom.

In yet another embodiment, the invention relates to compounds of formula I, wherein two substituents R⁵ are independently selected halogen atoms. Such halogen atoms are typically selected from the group consisting of fluoro and chloro atoms.

In yet another embodiment, the invention relates to compounds of formula I, wherein one R⁵ is cyano.

In yet another embodiment, the invention relates to compounds of formula I, wherein one R⁵ is C₁₋₆-alk(en/yn)yl.

In yet another embodiment, the invention relates to compounds of formula I, wherein one R⁵ is halo-C₁₋₆-alk(en/yn)yl, typically trifluoromethyl.

In yet another embodiment, the invention relates to compounds of formula I, wherein one R⁵ is C₃₋₈-cycloalk(en)yl.

In yet another embodiment, the invention relates to compounds of formula I, wherein one R⁵ is Ar, typically phenyl.

In yet another embodiment, the invention relates to compounds of formula I, wherein one R⁵ is C₁₋₆-alk(en/yn)yloxy.

In yet another embodiment, the invention relates to compounds of formula I, wherein two adjacent R⁵ form a 5-8 membered saturated carbocyclic ring.

In yet another embodiment, the invention relates to compounds of formula I, with the proviso that when X is CO, q is 0 and R³ is C₁₋₆-alk(en/yn)yl such as a methyl group then s is not 0 when R² is a hydrogen atom.

In yet another embodiment, the invention relates to compounds of formula I, with the proviso that when X is CO, q is 0 and R³ is C₁₋₆-alk(en/yn)yl such as a methyl group, then R² is different from a hydrogen atom when s is 1.

In yet another embodiment, the invention relates to compounds of formula I, with the proviso that when s is 0 and R² is a hydrogen atom then NH—X-(Z)_{q}-R³ is not acetamide.

In yet another embodiment, the compound of formula I is not:
N-[4-[[(4-aminophenyl)amino]methyl]phenyl]-acetamide;
N-[4-[[(4-amino-2-methylphenyl)amino]methyl]phenyl]-acetamide;
N-[4-[[(4-amino-3-methylphenyl)amino]methyl]phenyl]-acetamide;
2-[[[4-(acetylamino)phenyl]methyl]amino]-5-chloro-N-(5-chloro-2-pyridinyl)-benzamide;
N-[4-[[(3,4,5-trimethoxyphenyl)amino]methyl]phenyl]-acetamide;
N-[4-[[(5,6,7,8-tetrahydro-5,5,8,8-tetramethyl-2-naphthalenyl)amino]methyl]phenyl]-acetamide;
N-[4-[[[3-(1H-imidazol-1-ylmethyl)phenyl]amino]methyl]phenyl]-acetamide;
N-[4-[[[2-(1H-imidazol-1-ylmethyl)phenyl]amino]methyl]phenyl]-acetamide;
N-[4-[[[4-(1H-imidazol-1-ylmethyl)phenyl]amino]methyl]phenyl]-acetamide;
N-[4-[[(4-amino-3,5-dichlorophenyl)amino]methyl]phenyl]-acetamide;
N-[4-[[(2,4-diamino-6-quinazolinyl)amino]methyl]phenyl]-acetamide; or
N-[4-[[(2,4-diamino-6-quinazolinyl)amino]methyl]phenyl]-acetamide.

In yet another embodiment, the compound of formula I is not:
2-[[[4-(acetylamino)phenyl]methyl]amino]-5-chloro-N-(5-chloro-2-pyridinyl)-benzamide;
N-[4-[[(3,4,5-trimethoxyphenyl)amino]methyl]phenyl]-acetamide;
N-[4-[[(5,6,7,8-tetrahydro-5,5,8,8-tetramethyl-2-naphthalenyl)amino]methyl]phenyl]-acetamide;
N-[4-[[[3-(1H-imidazol-1-ylmethyl)phenyl]amino]methyl]phenyl]-acetamide;
N-[4-[[[2-(1H-imidazol-1-ylmethyl)phenyl]amino]methyl]phenyl]-acetamide;
N-[4-[[[4-(1H-imidazol-1-ylmethyl)phenyl]amino]methyl]phenyl]-acetamide;
N-[4-[[(4-amino-3,5-dichlorophenyl)amino]methyl]phenyl]-acetamide;
N-[4-[[(2,4-diamino-6-quinazolinyl)amino]methyl]phenyl]-acetamide; or
N-[4-[[(2,4-diamino-6-quinazolinyl)amino]methyl]phenyl]-acetamide.

The molecular weight of the compounds of the invention may vary from compound to compound. The molecular weight of a compound of formula I is typically more than 200 and less than 600, and more typically more than 250 and less than 550.

One aspect of the invention, relates to compounds of general formula XI and salts thereof: 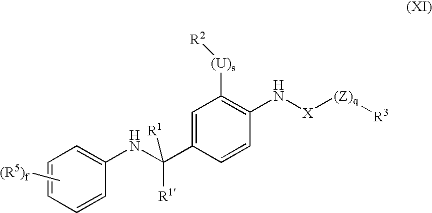
wherein f, s, q, U, X, Z, R¹, R^{1′}, R², R³ and R⁵ are as defined above, accordingly any of f, s, q, U, X, Z, R¹, R^{1′}, R², R^{2′}, R³, R⁴, R⁵, R⁶, R^{6′}, R⁷, R^{7′}, R⁸, R⁹ and R^{9′} are as defined under formula I. Any of the embodiments related to formula I are also embodiments of formula XI.

In one embodiment, the invention relates to compounds of the general formula XI, which is not substituted by R⁵.

In another embodiment, the invention relates to compounds of the general formula XI being monosubstituted by R⁵, such as in the ortho-, meta- or para-position.

In yet another embodiment, the invention relates to compounds of the general formula XI being disubstituted by R⁵, such as in the ortho- and para-position, in the meta- and para-position and in the ortho- and meta-position.

In yet another embodiment, the invention relates to compounds of the general formula XI being trisubstituted by R⁵.

Another aspect of the invention relates to compounds of the general formula XII or salts thereof: 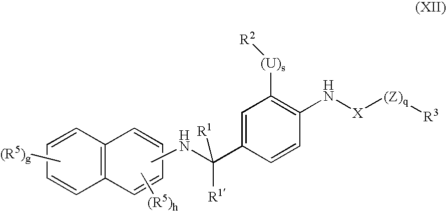
wherein g, h, s, q, U, X, Z, R¹, R^{1′}, R², R³ and R⁵ are as defined above, accordingly any of g, h, s, q, U, X, Z, R¹, R^{1′}, R², R^{2′}, R³, R⁴, R⁵, R⁶, R^{6′}, R⁷, R^{7′}, R⁸, R⁹ and R^{9′} are as defined under formula I. Any of the embodiments related to formula I are also embodiments of formula XII.

In one embodiment, the invention relates to compounds of the general formula XII, wherein the nitrogen atom is attached to position 1 of the naphtyl group.

In another embodiment, the invention relates to compounds of the general formula XII, wherein the nitrogen atom is attached to position 2 of the naphtyl group.

In yet another embodiment, the invention relates to compounds of the general formula XII, wherein g is 0, 1, 2 or 3, typically 0, 1 or 2.

In yet another embodiment, the invention relates to compounds of the general formula XII, wherein h is 0, 1 or 2, typically 0 or 1.

In yet another embodiment, the invention relates to compounds of the general formula XII, which are not substituted by R⁵.

In yet another embodiment, the invention relates to compounds of the general formula XII being monosubstituted by R⁵.

In yet another embodiment, the invention relates to compounds of the general formula XII being disubstituted by R⁵.

In yet another embodiment, the invention relates to compounds of the general formula XII being trisubstituted by R⁵.

Yet another aspect of the invention relates to compounds of the general formula XIII or salts thereof: 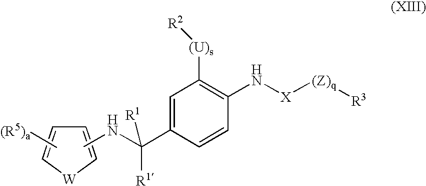
wherein a, s, q, U, W, X, Z, R¹, R^{1′}, R², R³ and R⁵ are as defined above, accordingly any of a, s, q, U, W, X, Z, R¹, R^{1′}, R², R^{2′}, R³, R⁴, R⁵, R⁶, R^{6′}, R⁷, R^{7′}, R⁸, R⁹ and R^{9′} are as defined under formula I. Any of the embodiments related to formula I are also embodiments of formula XIII.

In one embodiment, the invention relates to compounds of the general formula XIII, wherein the nitrogen atom is attached to position 2 of the heteroaromatic group.

In another embodiment, the invention relates to compounds of the general formula XIII, wherein the nitrogen atom is attached to position 3 of the heteroaromatic group.

In yet another embodiment, the invention relates to compounds of the general formula XIII, wherein a is 0, 1 or 2.

In yet another embodiment, the invention relates to compounds of the general formula XIII, which are not substituted by R⁵.

In yet another embodiment, the invention relates to compounds of the general formula XIII being monosubstituted by R⁵.

In yet another embodiment, the invention relates to compounds of the general formula XIII being disubstituted by R⁵.

Yet another aspect of the invention relates to compounds of the general formula XIV or salts thereof: 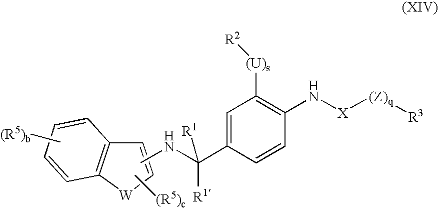
wherein b, c, s, q, U, W, X, Z, R¹, R^{1′}, R², R³ and R⁵ are as defined above, accordingly any of b, c, s, q, U, W, X, Z, R¹, R^{1′}, R², R^{2′}, R³, R⁴, R⁵, R⁶, R^{6′}, R⁷, R^{7′}, R⁸, R⁹ and R^{9′} are as defined under formula I. Any of the embodiments related to formula I are also embodiments of formula XIV.

In one embodiment, the invention relates to compounds of the general formula XIV, wherein the nitrogen atom is attached to position 2 of the heteroaromatic group.

In another embodiment, the invention relates to compounds of the general formula XIV, wherein the nitrogen atom is attached to position 3 of the heteroaromatic group.

In yet another embodiment, the invention relates to compounds of the general formula XIV, wherein b is 0, 1, 2 or 3, typically 0, 1 or 2.

In yet another embodiment, the invention relates to compounds of the general formula XIV, wherein c is 0 or 1, typically 0.

In yet another embodiment, the invention relates to compounds of the general formula XIV, which is not substituted by R⁵.

In yet another embodiment, the invention relates to compounds of the general formula XIV being monosubstituted by R⁵.

In yet another embodiment, the invention relates to compounds of the general formula XIV being disubstituted by R⁵.

In yet another embodiment, the invention relates to compounds of the general formula XIV being trisubstituted by R⁵.

Yet another aspect of the invention relates to compounds of the general formula XV or salts thereof: 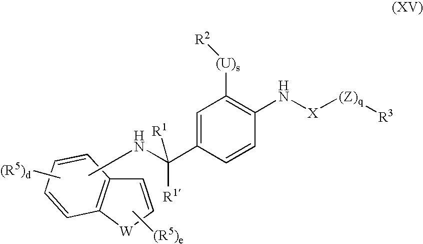
wherein d, e, s, q, U, W, X, Z, R¹, R^{1′}, R², R³ and R⁵ are as defined above, accordingly d, e, s, q, U, W, X, Z, R¹, R^{1′}, R², R^{2′}, R³, R⁴, R⁵, R⁶, R^{6′}, R⁷, R^{7′}, R⁸, R⁹ and R^{9′} are as defined under formula I. Any of the embodiments related to formula I are also embodiments of formula XV.

In one embodiment, the invention relates to compounds of the general formula XV, wherein the nitrogen atom is attached to position 4 of the heteroaromatic group.

In another embodiment, the invention relates to compounds of the general formula XV, wherein the nitrogen atom is attached to position 5 of the heteroaromatic group.

In one embodiment, the invention relates to compounds of the general formula XV, wherein the nitrogen atom is attached to position 6 of the heteroaromatic group.

In another embodiment, the invention relates to compounds of the general formula XV, wherein the nitrogen atom is attached to position 7 of the heteroaromatic group.

In yet another embodiment, the invention relates to compounds of the general formula XV, wherein d is 0, 1 or 2, typically 0 or 1.

In yet another embodiment, the invention relates to compounds of the general formula XV, wherein e is 0 or 1.

In yet another embodiment, the invention relates to compounds of the general formula XV, which is not substituted by R⁵.

In yet another embodiment, the invention relates to compounds of the general formula XV being monosubstituted by R⁵.

In yet another embodiment, the invention relates to compounds of the general formula XV being disubstituted by R⁵.

In yet another embodiment, the invention relates to compounds of the general formula XV being trisubstituted by R⁵.

The compounds of the following list and salts thereof are preferred:
{2-Amino-4-[(4-tert-butylphenylamino)-methyl]-phenyl}-carbamic acid ethyl ester;
(2-Amino-4-phenylaminomethyl-phenyl)-carbamic acid ethyl ester;
[2-Amino-4-(naphthalen-2-ylaminomethyl)-phenyl]-carbamic acid ethyl ester;
[2-Amino-4-(p-tolylamino-methyl)-phenyl]-carbamic acid ethyl ester;
{2-Amino-4-[(4-trifluoromethylphenylamino)-methyl]-phenyl}-carbamic acid ethyl ester;
{2-Amino-4-[(4-chlorophenylamino)-methyl]-phenyl}-carbamic acid ethyl ester;
{2-Amino-4-[(3-fluorophenylamino)-methyl]-phenyl}-carbamic acid ethyl ester;
{2-Amino-4-[(4-fluorophenylamino)-methyl]-phenyl}-carbamic acid ethyl ester;
{2-Amino-4-[(2-fluorophenylamino)-methyl]-phenyl}-carbamic acid ethyl ester;
[2-Amino-4-(biphenyl-4-ylaminomethyl)-phenyl]-carbamic acid ethyl ester;
{2-Amino-4-[(2,4-difluorophenylamino)-methyl]-phenyl}-carbamic acid ethyl ester;
{2-Amino-4-[(4-methoxyphenylamino)-methyl]-phenyl}-carbamic acid ethyl ester;
{2-Amino-4-[(4-cyclohexylphenylamino)-methyl]-phenyl}-carbamic acid ethyl ester;
[2-Amino-4-(indan-5-ylaminomethyl)-phenyl]-carbamic acid ethyl ester;
{2-Amino-4-[(4-isopropylphenylamino)-methyl]-phenyl}-carbamic acid ethyl ester; and
{2-Amino-4-[(4-butylphenylamino)-methyl]-phenyl}-carbamic acid ethyl ester.

In another embodiment, the compounds of the following list and salts thereof are preferred:
{2-Amino-4-[(4-tert-butylphenylamino)-methyl]-phenyl}-carbamic acid ethyl ester;
(2-Amino-4-phenylaminomethyl-phenyl)-carbamic acid ethyl ester;
[2-Amino-4-(naphthalen-2-ylaminomethyl)-phenyl]-carbamic acid ethyl ester;
[2-Amino-4-(p-tolylamino-methyl)-phenyl]-carbamic acid ethyl ester;
{2-Amino-4-[(4-trifluoromethylphenylamino)-methyl]-phenyl}-carbamic acid ethyl ester;
{2-Amino-4-[(4-chlorophenylamino)-methyl]-phenyl}-carbamic acid ethyl ester;
{2-Amino-4-[(3-fluorophenylamino)-methyl]-phenyl}-carbamic acid ethyl ester;
{2-Amino-4-[(4-fluorophenylamino)-methyl]-phenyl}-carbamic acid ethyl ester;
{2-Amino-4-[(2-fluorophenylamino)-methyl]-phenyl}-carbamic acid ethyl ester;
[2-Amino-4-(biphenyl-4-ylaminomethyl)-phenyl]-carbamic acid ethyl ester;
{2-Amino-4-[(2,4-difluorophenylamino)-methyl]-phenyl}-carbamic acid ethyl ester;
{2-Amino-4-[(4-methoxyphenylamino)-methyl]-phenyl}-carbamic acid ethyl ester;
{2-Amino-4-[(4-cyclohexylphenylamino)-methyl]-phenyl}-carbamic acid ethyl ester;
[2-Amino-4-(indan-5-ylaminomethyl)-phenyl]-carbamic acid ethyl ester;
{2-Amino-4-[(4-isopropylphenylamino)-methyl]-phenyl}-carbamic acid ethyl ester;
{2-Amino-4-[(4-butylphenylamino)-methyl]-phenyl}-carbamic acid ethyl ester;
{2-Amino-4-[(4-chloro-3-fluorophenylamino)-methyl]phenyl}carbamic acid ethyl ester;
{2-Amino-4-[(2,4-dichlorophenylamino)methyl]phenyl}carbamic acid ethyl ester;
{2-Amino-4-[(2,3-dichlorophenylamino)methyl]phenyl}carbamic acid ethyl ester;
{2-Amino-4-[(3,5-dichlorophenylamino)methyl]phenyl}carbamic acid ethyl ester;
{2-Amino-4-[(3,4-dichlorophenylamino)methyl]phenyl}carbamic acid ethyl ester;
{2-Amino-4-[(3-trifluoromethylphenylamino)methyl]phenyl}carbamic acid ethyl ester;
{2-Amino-4-[(3-fluoro-4-trifluoromethylphenylamino)methyl]phenyl}carbamic acid ethyl ester;
{2-Amino-4-[(3,4-difluorophenylamino)methyl]phenyl}carbamic acid ethyl ester;
{2-Amino-4-[(4-cyanophenylamino)methyl]phenyl}carbamic acid ethyl ester;
{2-Amino-4-[(4-fluoro-3-trifluoromethylphenylamino)methyl]phenyl}carbamic acid ethyl ester;
{2-Amino-4-[(3-chloro-4-methylphenylamino)methyl]phenyl}carbamic acid ethyl ester;
{2-Amino-4-[(3-chlorophenylamino)methyl]phenyl}carbamic acid ethyl ester;
[2-Amino-4-(m-tolylaminomethyl)phenyl]carbamic acid ethyl ester;
{2-Amino-4-[1-(4-chlorophenylamino)ethyl]phenyl}carbamic acid ethyl ester;
{2-Amino-4-[1-(4-trifluoromethylphenylamino)ethyl]phenyl}carbamic acid ethyl ester;
N-{2-Amino-4[(3-fluorophenylamino)methyl]phenyl}-2,2-dimethylpropionamide;
{4-[(4-Chlorophenylamino)methyl]phenyl}carbamic acid ethyl ester;
{4-[(4-Trifluoromethylphenylamino)methyl]phenyl}carbamic acid ethyl ester;
{4-[1-(4-Chlorophenylamino)ethyl]phenyl}carbamic acid ethyl ester;
{4-[(4-Fluorophenylamino)methyl]-2-methylphenyl}carbamic acid ethyl ester;
{4-[(4-Chlorophenylamino)methyl]-2-methylphenyl}carbamic acid ethyl ester;
{2-Methyl-4-[(4-trifluoromethylphenylamino)methyl]phenyl}carbamic acid ethyl ester;
{4-[(3,4-Difluorophenylamino)methyl]-2-methylphenyl}carbamic acid ethyl ester;
{4-[(3-Fluorophenylamino)methyl]-2-methylphenyl}carbamic acid ethyl ester;
{2-Chloro-4-[(4-chlorophenylamino)methyl]phenyl}carbamic acid ethyl ester;
{2-Chloro-4-[(4-trifluoromethyl-phenylamino)-methyl]-phenyl}-carbamic acid ethyl ester;
{2-Chloro-4-[(4-fluorophenylamino)methyl]phenyl}carbamic acid ethyl ester;
{2-Chloro-4-[(3-fluorophenylamino)methyl]phenyl}carbamic acid ethyl ester;
{2-Chloro-4-[(3,4-dichlorophenylamino)methyl]phenyl}carbamic acid ethyl ester;
{2-Chloro-4-[(4-chloro-3-fluorophenylamino)methyl]phenyl}carbamic acid ethyl ester;
{4-[(4-Chlorophenylamino)methyl]-2-fluorophenyl}carbamic acid ethyl ester;
{4-[(4-Chloro-3-fluorophenylamino)methyl]-2-fluorophenyl}carbamic acid ethyl ester;
{2-Fluoro-4-[(4-trifluoromethylphenylamino)methyl]phenyl}carbamic acid ethyl ester;
{4′-Dimethylamino-5-[(3-fluorophenylamino)methyl]biphenyl-2-yl}carbamic acid ethyl ester;
{4′-Dimethylamino-5-[(4-trifluoromethylphenylamino)methyl]biphenyl-2-yl}carbamic acid ethyl ester;
{4′-Chloro-5-[(3-fluorophenylamino)methyl]biphenyl-2-yl}carbamic acid ethyl ester;
{4′-Chloro-5-[(4-trifluoromethylphenylamino)methyl]biphenyl-2-yl}carbamic acid ethyl ester;
N-{4-[(4-chlorophenylamino)methyl]phenyl}butyramide;
N-{4-[(3,4-dichlorophenylamino)methyl]phenyl}butyramide;
N-{4-[(4-chloro-3-fluorophenylamino)methyl]phenyl}butyramide;
N-{4[(4-fluoro-phenylamino)methyl]-2-methylphenyl}butyramide;
N-{4[(3-fluorophenylamino)methyl]-2-methylphenyl}butyramide;
N-{4-[(4-chlorophenylamino)methyl]-2-methylphenyl}butyramide;
N-{4-[(3,4-dichlorophenylamino)methyl]-2-methylphenyl}butyramide;
N-{4-[(4-chloro-3-fluorophenylamino)methyl]-2-methylphenyl}butyramide;
N-{2-chloro-4-[(4-trifluoromethylphenylamino)methyl]phenyl}butyramide;
N-{2-chloro-4-[(4-fluorophenylamino)methyl]phenyl}butyramide;
N-{2-chloro-4-[(3-fluorophenylamino)methyl]phenyl}butyramide;
N-{2-chloro-4-[(4-chlorophenylamino)methyl]phenyl}butyramide;
N-{2-chloro-4-[(3,4-dichlorophenylamino)methyl]phenyl}butyramide;
N-{2-chloro-4-[(4-chloro-3-fluorophenylamino)methyl]phenyl}butyramide;
N-{2-fluoro-4-[(3-fluorophenylamino)methyl]phenyl}butyramide;
N-{4-[(4-chlorophenylamino)methyl]-2-fluorophenyl}butyramide;
N-{2-fluoro-4-[(4-trifluoromethylphenylamino)methyl]phenyl}butyramide,
N-{4-[(3,4-dichlorophenylamino)methyl]-2-fluorophenyl}butyramide; and
N-{4-[(4-chloro-3-fluorophenylamino)methyl]-2-fluorophenyl}butyramide.

According to one embodiment, the invention relates to a pharmaceutical composition comprising one or more pharmaceutically acceptable carriers or diluents and a compound of formula I wherein Y, U, X, Z, s, q, R¹, R^{1′}, R² and R³ are as defined above, accordingly any of s, q, U, X, Z, Y, W, R⁴, R¹, R^{1′}, R², R^{2′}, R³, R⁴, R⁶, R^{6′}, R⁷, R^{7′}, R⁸, R⁹ and R^{9′} are as defined under formula I, or salts thereof. Pharmaceutical compositions of the invention may thus comprise one or more compounds of formula I or salts thereof, such as one compound of formula I or a salt thereof; or two compounds of formula I or salts thereof; or three compounds of formula I or salts thereof.

According to another embodiment, the invention relates to a pharmaceutical composition comprising one or more pharmaceutically acceptable carriers or diluents and a compound of formula XI wherein f, s, q, U, X, Z, R¹, R^{1′}, R², R³ and R⁵ are as defined above, accordingly any of f, s, q, U, X, Z, R¹, R^{1′}, R², R^{2′}, R³, R⁴, R⁵, R⁶, R⁶′, R⁷, R^{7′}, R⁸, R⁹ and R^{9′} are as defined under formula XI, or salts thereof. Pharmaceutical compositions of the invention may thus comprise one or more compounds of formula XI or salts thereof, such as one compound of formula XI or a salt thereof; or two compounds of formula XI or salts thereof; or three compounds of formula XI or salts thereof.

According to yet another embodiment, the invention relates to a pharmaceutical composition comprising one or more pharmaceutically acceptable carriers or diluents and a compound of formula XII wherein g, h, s, q, U, X, Z, R¹, R^{1′}, R², R³ and R⁵ are as defined above, accordingly any of g, h, s, q, U, X, Z, R¹, R^{1′}, R², R^{2′}, R³, R⁴, R⁵, R⁶, R^{6′}, R⁷, R^{7′}, R⁸, R⁹ and R^{9′} are as defined under formula XII, or salts thereof. Pharmaceutical compositions of the invention may thus comprise one or more compounds of formula XII or salts thereof, such as one compound of formula XII or a salt thereof; or two compounds of formula XII or salts thereof; or three compounds of formula XI or salts thereof.

According to yet another embodiment, the invention relates to a pharmaceutical composition comprising one or more pharmaceutically acceptable carriers or diluents and a compound of formula XIII wherein a, s, q, U, W, X, Z, R¹, R^{1′}, R², R³ and R⁵ are as defined above, accordingly any of a, s, q, U, X, Z, W, R¹, R^{1′}, R², R^{2′}, R³, R⁴, R⁵, R⁶, R^{6′}, R⁷, R^{7′}, R⁸, R⁹ and R^{9′} are as defined under formula XIII, or salts thereof. Pharmaceutical compositions of the invention may thus comprise one or more compounds of formula XIII or salts thereof, such as one compound of formula XIII or a salt thereof; or two compounds of formula XIII or salts thereof; or three compounds of formula XIII or salts thereof.

According to yet another embodiment, the invention relates to a pharmaceutical composition comprising one or more pharmaceutically acceptable carriers or diluents and a compound of formula XIV wherein b, c, s, q, U, W, X, Z, R¹, R^{1′}, R², R³ and R⁵ are as defined above, accordingly any of b, c, s, q, U, X, Z, W, R¹, R^{1′}, R², R^{2′}, R³, R⁴, R⁵, R⁶, R^{6′}, R⁷, R^{7′}, R⁸, R⁹ and R^{9′} are as defined under formula XIV, or salts thereof. Pharmaceutical compositions of the invention may thus comprise one or more compounds of formula XIV or salts thereof, such as one compound of formula XIV or a salt thereof; or two compounds of formula XIV or salts thereof, or three compounds of formula XIV or salts thereof.

According to yet another embodiment, the invention relates to a pharmaceutical composition comprising one or more pharmaceutically acceptable carriers or diluents and a compound of formula XV wherein d, e, s, q, U, W, X, Z, R¹, R^{1′}, R², R³ and R⁵ are as defined above, accordingly d, e, s, q, U, X, Z, W, R¹, R^{1′}, R², R^{2′}, R³, R⁴, R⁵, R⁶, R^{6′}, R⁷, R^{7′}, R⁸, R⁹ and R^{9′} are as defined under formula XV, or salts thereof. Pharmaceutical compositions of the invention may thus comprise one or more compounds of formula XV or salts thereof, such as one compound of formula XV or a salt thereof; or two compounds of formula XV or salts thereof; or three compounds of formula XV or salts thereof.

The invention provides a pharmaceutical composition for oral or parenteral administration, said pharmaceutical composition comprising at least one compound of formula I or a salt thereof in a therapeutically effective amount together with one or more pharmaceutically acceptable carriers or diluents.

In one aspect, the compounds of the invention may be administered as the only therapeutically effective compound.

In another aspect the compounds of the invention may be administered as a part of a combination therapy, i.e. the compounds of the invention may be administered in combination with other therapeutically effective compounds having e.g. anti-convulsive properties. The effects of such other compounds having anti-convulsive properties may include but not be limited to activities on:
ion channels such as sodium, potassium, or calcium channels
the excitatory amino acid systems e.g. blockade or modulation of NMDA receptors
the inhibitory neurotransmitter systems e.g. enhancement of GABA release, or blockade of GABA-uptake or
membrane stabilisation effects.

Current anti-convulsive medications include, but are not limited to, tiagabine, carbamazepine, sodium valproate, lamotrigine, gabapentin, pregabalin, ethosuximide, levetiracetam, phenytoin, topiramate, zonisamide as well as members of the benzodiazepine and barbiturate class.

In one aspect, the compounds of the invention have been found to have effect on potassium channels of the KCNQ family, in particular the KCNQ2 subunit.

In one embodiment, the invention relates to the use of one or more compounds according to the invention in a method of treatment. The disorder or condition to be prevented, treated or inhibited is responsive to an increased ion flow in a potassium channel such as the KCNQ family potassium ion channels. Such disorder or condition is preferably a disorder or condition of the central nervous system.

The compounds of the invention are considered useful for increasing ion flow in a voltage-dependent potassium channel in a mammal such as a human.

The compounds of the invention are considered useful for the prevention, treatment or inhibition of a disorder or condition being responsive to an increased ion flow in a potassium channel such as the KCNQ family potassium ion channels. Such disorder or condition is preferably a disorder or condition of the central nervous system.

The compounds of the invention are thus considered useful for preventing, treating or inhibiting disorders or diseases such as seizure disorders, neuropathic and migraine pain disorders, anxiety disorders and neurodegenerative disorders.

Accordingly, the compounds of the invention are considered useful for the prevention, treatment or inhibition of disorders or conditions such as convulsions, epilepsy, anxiety disorders, neuropathic pain and neurodegenerative disorders.

According to one particular aspect, the compounds of the invention are considered to be useful for preventing, treating or inhibiting seizure disorders such as convulsions, epilepsy and status epilepticus.

In one embodiment, the compounds of the invention are considered useful in the prevention, treatment and inhibition of convulsions.

In another embodiment, the compounds of the invention are considered useful in the prevention, treatment and inhibition of epilepsy, epileptic syndromes and epileptic seizures.

In yet another embodiment, the compounds of the invention are considered useful in the prevention, treatment and inhibition of anxiety disorders such as anxiety and conditions and diseases related to panic attack, agoraphobia, panic disorder with agoraphobia, panic disorder without agoraphobia, agoraphobia without history of panic disorder, specific phobia, social phobia and other specific phobias, obsessive-compulsive disorder, posttraumatic stress disorder, acute stress disorders, generalized anxiety disorder, anxiety disorder due to general medical condition, substance-induced anxiety disorder, separation anxiety disorder, adjustment disorders, performance anxiety, hypochondriacal disorders, anxiety disorder due to general medical condition and substance-induced anxiety disorder and anxiety disorder not otherwise specified.

In yet another embodiment, the compounds of the invention are considered useful in the prevention, treatment and inhibition of neuropathic pain and migraine pain disorders such as allodynia, hyperalgesic pain, phantom pain, neuropathic pain related to diabetic neuropathy and neupathic pain related to migraine.

In yet another embodiment, the compounds of the invention are considered useful in the prevention, treatment and inhibition of neurodegenerative disorders such as Alzheimer's disease; Huntington's chorea; multiple sclerosis; amyotrophic lateral sclerosis; Creutzfeld-Jakob disease; Parkinson's disease; encephalopathies induced by ADDS or infection by rubella viruses, herpes viruses, borrelia and unknown pathogens; trauma-induced neurodegenerations; neuronal hyperexcitation states such as in medicament withdrawal or intoxication; and neurodegenerative diseases of the peripheral nervous system such as polyneuropathies and polyneuritides.

In yet another embodiment, the compounds of the invention are considered useful in the prevention, treatment and inhibition of neurodegenerative disorders such as Alzheimer's disease; Huntington's chorea; multiple sclerosis; amyotrophic lateral sclerosis; Creutzfeld-Jakob disease; Parkinson's disease; encephalopathies induced by AIDS or infection by rubella viruses, herpes viruses, borrelia and unknown pathogens; and trauma-induced neurodegenerations.

In yet another embodiment, the compounds of the invention are considered useful in the prevention, treatment and inhibition of neuronal hyperexcitation states such as in medicament withdrawal or intoxication.

The invention provides compounds showing effect in one or more of the following tests:
“Relative efflux through the KCNQ2 channel”
Which is a measure of the potency of the compound at the target channel
“Maximum electroshock”
Which is a measure of seizures induced by non-specific CNS stimulation by electrical means
“Pilocarpine induced seizures”
Seizures induced by pilocarpine are often difficult to treat with many existing antiseizure medications and so reflect a model of “drug resistant seizures”
“Electrical seizure-threshold tests” and “Chemical seizure-threshold tests”
These models measure the threshold at which seizures are initiated, thus being models that detect whether compounds could delay seizure initiation.
“Amygdala kindling”
Which is used as a measure of disease progression, as in normal animals the seizures in this model get more severe as the animal receives further stimulations.

According to one particular aspect of the invention, the compounds are KCNQ2 active with an EC₅₀ of less than 15000 nM such as less than 10000 nM as measured by the test “Relative efflux through the KCNQ2 channel” which is described below. According to one particular aspect of the invention, the compounds are KCNQ2 active with an EC₅₀ of less than 200 nM such as less than 1500 nM as measured by the test “Relative efflux through the KCNQ2 channel” which is described below.

According to another particular aspect of the invention, the compounds are KCNQ2 active with an EC₅₀ of less than 200 nM such as less than 150 nM as measured by the test “Relative efflux through the KCNQ2 channel” which is described below.

According to another particular aspect of the invention, the compounds have an ED₅₀ of less than 15 mg/kg in the test “Maximum electroshock” which is described below.

According to yet another particular aspect of the invention, the compounds have an ED₅₀ of less than 5 mg/kg in the test “Maximum electroshock” which is described below.

According to one particular aspect of the invention, the compounds have an ED₅₀ of less than 5 mg/kg in the “Electrical seizure-threshold test” and “Chemical seizure-threshold test” which is described below.

Some compounds have few or clinically insignificant side effects. Some of the compounds are thus tested in models of the unwanted sedative, hypothermic and ataxic actions of the compounds.

Some of the compounds have a large therapeutic index between anticonvulsant efficacy and side-effects such as impairment of locomotor activity or ataxic effects as measured by performance on a rotating rod. This means that the compounds will expectedly be well tolerated in patients permitting high doses to be used before side effects are seen. Thereby compliance with the therapy will expectedly be good and administration of high doses may be permitted making the treatment more efficacious in patients who would otherwise have side effects with other medications.

Definitions

The term heteroatom refers to a nitrogen, oxygen or sulphur atom.

Halogen means fluoro, chloro, bromo or iodo.

The expressions C₁₋₆-alk(en/yn)yl and C₁₋₆alk(an/en/yn)yl mean a C₁₋₆alkyl, C₂₋₆-alkenyl or a C₂₋₆-alkynyl group.

The term C₁₋₆-alkyl refers to a branched or unbranched alkyl group having from one to six carbon atoms inclusive, including but not limited to methyl, ethyl, 1-propyl, 2-propyl, 1-butyl, 2-butyl, 2-methyl-2-propyl, 2-2-dimethyl-1-propyl and 2-methyl-1-propyl.

Similarly, C₂₋₆-alkenyl and C₂₋₆-alkynyl, respectively, designate such groups having from two to six carbon atoms, including one double bond and one triple bond respectively, including but not limited to ethenyl, propenyl, butenyl, ethynyl, propynyl and butynyl.

The expressions C₁₋₄alkyl and C₁₋₄-alkanyl refer to a branched or unbranched alkyl group having from one to four carbon atoms inclusive, including but not limited to methyl, ethyl, 1-propyl, 2-propyl, 1-butyl, 2-butyl, 2-methyl-2-propyl and 2-methyl-1-propyl.

The expression C₁₋₃-alk(en/yn)yl means a C₁₋₃-alkyl, C₂₋₃-alkenyl or a C₂₋₃-alkynyl group.

The term C₁₃-alkyl refers to a branched or unbranched alkyl group having from one to three carbon atoms inclusive, including but not limited to methyl, ethyl, 1-propyl and 2-propyl.

Similarly, C₂₋₃-alkenyl and C₂₋₃-alkynyl, respectively, designate such groups having from two to three carbon atoms, including one double bond and one triple bond respectively, including but not limited to ethenyl, propenyl, ethynyl and propynyl.

The expressions C₃₋₈-cycloalk(en)yl and C₃₋₈-cycloalk(an/en)yl mean a C₃₋₈-cycloalkyl- or cycloalkenyl group.

The term C₃₋₈-cycloalkyl designates a monocyclic or bicyclic carbocycle having three to eight C-atoms, including but not limited to cyclopropyl, cyclopentyl, cyclohexyl, etc.

The expressions C₃₋₆-cycloalk(en)yl and C₃₋₆-cycloalk(an/en)yl mean a C₃₋₆-cycloalkyl- or cycloalkenyl group.

The term C₃₋₆-cycloalkyl designates a monocyclic or bicyclic carbocycle having three to six C-atoms, including but not limited to cyclopropyl, cyclopentyl, cyclohexyl, etc.

The term C₃₋₈-cycloalkenyl designates a monocyclic or bicyclic carbocycle having three to eight C-atoms and including one double bond.

The expression C₅₋₈-cycloalk(en)yl means a C₅₋₈-cycloalkyl- or cycloalkenyl group.

The term C₅₋₈-cycloalkyl designates a monocyclic or bicyclic carbocycle having five to eight C-atoms, including but not limited to cyclopentyl, cyclohexyl, etc.

The term C₅₋₈-cycloalkenyl designates a monocyclic or bicyclic carbocycle having five to eight C-atoms and including one or two double bonds.

In the term C₃₋₈-cycloalk(en)yl-C₁₋₆-alk(en/yn)yl, C₃₋₈-cycloalk(en)yl and C₁₋₆-alk(en/yn)yl are as defined above.

The term Ar refers to optionally substituted aromatic systems of 5-10 carbon atoms, wherein 0, 1, 2, 3 or 4 carbon atoms may be replaced with independently selected heteroatoms. Examples of such Ar groups are optionally substituted phenyl, optionally substituted naphtyl, optionally substituted thiophene, optionally substituted furan, optionally substituted thiazole, optionally substituted pyridine, optionally substituted pyrimidine, optionally substituted pyrrole and optionally substituted oxazole. Ar may be substituted with one or more substituents independently being hydroxy, halogen, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, C₁₋₆-alk(an/en/yn)yloxy, C₃₋₈-alk(an/en/yn)yloxy, acyl, cyano, —CO—NH—C₁₋₆-alk(en/yn)yl, —CO—N(C₁₋₆-alk(en/yn)yl)₂, —NH—C₁₋₆-alk(en/yn)yl, —N(C₁₋₆-alk(en/yn)yl)₂, —NH₂, —S—C₁₋₆-alk(en/yn)yl, —SO₂—C₁₋₆-alk(en/yn)yl, —SO₂N(C₁₋₆-alk(en/yn)yl)₂, —SO₂NH—C₁₋₆-alk(en/yn)yl and —SO₂O—C₁₋₆-alk(en/yn)yl; or two substituents may together form a 5-8 membered saturated or unsaturated ring which optionally contains one or two heteroatoms. Such two ring forming substituents may be adjacent and may together form:

—(CH₂)_{n**}—CH₂—, —CH═CH—(CH₂)_{m**}—, —CH₂—CH═CH—(CH₂)_{p**}, —(CH₂)_{n**}—O—, —O—(CH₂)_{m**}—O—, —CH₂—O—(CH₂)_{p**}—O—, —CH₂—O—CH₂—O—CH₂—, —(CH₂)_{n**}—S—, —S—(CH₂)_{m**}—S—, —CH₂—S—(CH₂)_{p**}—S—, —CH₂—S—CH₂—S—CH₂—, —(CH₂)_{n**}—NH—, —NH—(CH₂)_{m**}—NH—, —CH₂—NH—(CH₂)_{p**}—NH—, —CH═CH—NH—, —O—(CH₂)_{m**}—NH—, —CH₂—O—(CH₂)_{p**}—NH— or —O—(CH₂)_{p**}—NH—CH₂—, —S—(CH₂)_{m**}—NH—, —N═CH—NH—, —N═CH—O— or —N═CH—S—, wherein m** is 1, 2 or 3, n** is 2, 3 or 4 and p** is 1 or 2.

As used herein, the term acyl refers to formyl, C₁₋₆-alk(en/yn)ylcarbonyl, C₃₋₈-cycloalk(en)ylcarbonyl, Ar-carbonyl, Ar—C₁₋₆-alk(en/yn)ylcarbonyl or a C₃₋₈-cycloalk(en)yl-C₁₋₆-alk(en/yn)yl-carbonyl group, wherein C₁₋₆-alk(en/yn)yl, C₃₋₈-cycloalk(en)yl and Ar are as defined above.

The term halo-C₁₋₆-alk(en/yn)yl designates C₁₋₆-alk(en/yn)yl being substituted with one or more halogen atoms, including but not limited to trifluoromethyl. Similarly, halo-C₃₋₈-cycloalk(en)yl designates C₃₋₈-cycloalk(en)yl being substituted with one or more halogen atoms and halo-C₃₋₈-cycloalk(en)yl-C₁₋₆-alk(en/yn)yl, designates C₃₋₈-cycloalk(en)yl-C₁₋₆-alk(en/yn)yl being substituted with one or more halogen atoms.

The terms hydroxy-C₁₋₆-alk(en/yn)yl, hydroxy-C₃₋₈-alk(en/yn)yl, hydroxy-C₃₋₈-cycloalk(en)yl, Ar—C₁₋₆-alk(en/yn)yl, Ar—C₃₋₈-cycloalk(en)yl, C₁₋₆-alk(an/en/yn)yloxy, C₁₋₄-alkanyloxy, C₂₋₆-alkenyloxy, C₂₋₆-alkynyloxy, C₃₋₈-alk(an/en/yn)yloxy, C₁₋₆-alk(en/yn)ylcarbonyl, C₃₋₈-alk(en/yn)ylcarbonyl, Ar-carbonyl, Ar—C₁₋₆-alk(en/yn)ylcarbonyl, C₃₋₈-cycloalk(en)yl-C₁₋₆-alk(en/yn)ylcarbonyl etc. designate such groups in which the C₁₋₆-alk(en/yn)yl, C₂₋₆-alkenyl, C₂₋₆-alkynyl, C₃₋₈-cycloalk(en)yl and Ar are as defined above.

The term “two substituents together form a 5-8 membered saturated or unsaturated ring, which optionally contains one or two heteroatoms,” refers to aliphatic or aromatic carbocyclic or heterocyclic systems wherein the ring is formed by 5 to 8 atoms which may be substituted by one or more substituents independently being C₁₋₆-alk(en/yn)yl, C₃₋₈-alk(en/yn)yl, C₃₋₈-cycloalk(en)yl-C₁₋₆-alk(en/yn)yl, halogen, halo-C₁₋₆-alk(en/yn)yl, halo-C₃₋₈-alk(en/yn)yl or C₃₋₇-cycloalk(en)yl-C₁₋₆-alk(en/yn)yl. The ring forming atoms are selected from 3-8 carbon atoms and 0-2 heteroatoms selected from N, S, or O. When the two ring forming substituents are attached to the same nitrogen atom, then said nitrogen atom becomes one of the atoms forming the ring. When two ring forming substituents are attached to an aliphatic or aromatic carbocyclic or heterocyclic group, then the two ring forming substituents are conveniently attached adjacent to each other and the ring formed by the two substituents is fused to the aliphatic or aromatic carbocyclic or heterocyclic group. Two ring forming substituents may together be represented by:

—(CH₂)_{n″}—CH₂—, —CH═CH—(CH₂)_{m″}—, —CH₂—CH═CH—(CH₂)_{p″}, —CH═CH—CH═CH—, (CH₂)_{n″}—O—, —O—(CH₂)_{m″}—O—, —CH₂—O—(CH₂)_{p″}—O—, —CH₂—O—CH₂—O—CH₂—, —(CH₂)_{n″}—S—, —S—(CH₂)_{m″}—S—, —CH₂—S—(CH₂)_{p″}—S—, —CH₂—S—CH₂—S—CH₂—, —(CH₂)_{n″}—NH—, —NH—(CH₂)_{m″}—NH—, —CH₂—NH—(CH₂)_{p″}—NH—, —CH═CH—NH—, O—(CH₂)_{m″}—NH—, —CH₂——O—(CH₂)_{p″}—NH— or —O—(CH₂)_{p″}—NH—CH₂—, —S—(CH₂)_{m″}—NH—, —N═CH—NH—, —N═CH—O— or —N═CH—S—, wherein m″ is 1, 2 or 3, n″ is 2, 3 or 4 and p″ is 1 or 2.

The salts of the invention are preferably pharmaceutically acceptable salts. Such salts include pharmaceutical acceptable acid addition salts, pharmaceutically acceptable metal salts, ammonium and alkylated ammonium salts.

The pharmaceutically acceptable salts of the invention are preferably acid addition salts.

Acid addition salts include salts of inorganic acids as well as organic acids.

The acid addition salts of the invention are preferably pharmaceutically acceptable salts of the compounds of the invention formed with non-toxic acids.

Representative examples of suitable inorganic acids include hydrochloric, hydrobromic, hydroiodic, sulfuric, sulfamic, phosphoric and nitric acids and the like. Such acid addition salts can be formed by methods known to the person skilled in the art. Further examples of pharmaceutically acceptable inorganic or organic acid addition salts include the pharmaceutically acceptable salts listed in J. Pharm. Sci. 1977, 66, 2, which is incorporated herein by reference.

Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, ethanesulfonic, tartaric, ascorbic, pamoic, gluconic, citraconic, aspartic, stearic, palmitic, EDTA, glycolic, p-aminobenzoic, glutamic, bis-methylenesalicylic, methanesulfonic, ethanedisulfonic, itaconic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in J. Pharm. Sci. 1977, 66,2, which is incorporated herein by reference.

Examples of metal salts include lithium, sodium, potassium, magnesium salts and the like.

Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

Also intended as pharmaceutical acceptable acid addition salts are the hydrates, which the present compounds are able to form.

The compounds of the present invention may have one or more asymmetric centres and it is intended that any optical isomers, as separated, pure or partially purified optical isomers or racemic mixtures thereof are included within the scope of the invention.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms that the compounds are able to form are included within the scope of the present invention.

The compounds of this invention may exist in unsolvated as well as in solvated forms with solvents such as water, ethanol and the like. In general, the solvated forms are considered equivalent to the unsolvated forms for the purposes of this invention.

Some of the compounds of the present invention contain chiral centres and such compounds exist in the form of isomers (i.e. enantiomers). The invention includes all such isomers and any mixtures thereof including racemic mixtures.

Racemic forms can be resolved into the optical antipodes by known methods, for example, by separation of diastereomeric salts thereof with an optically active acid, and liberating the optically active amine compound by treatment with a base. Another method for resolving racemates into the optical antipodes is based upon chromatography on an optically active matrix. Racemic compounds of the present invention can also be resolved into their optical antipodes, e.g. by fractional crystallization of d- or 1-(tartrates, mandelates or camphorsulphonate) salts. The compounds of the present invention may also be resolved by the formation of diastereomeric derivatives.

Additional methods for the resolution of optical isomers, known to those skilled in the art, may be used. Such methods include those discussed by J. Jaques, A. Collet and S. Wilen in “Enantiomers, Racemates, and Resolutions”, John Wiley and Sons, New York (1981).

Optically active compounds can also be prepared from optically active starting materials.

The invention also encompasses prodrugs of the present compounds, which on administration undergo chemical conversion by metabolic processes before becoming pharmacologically active substances. In general, such prodrugs will be functional derivatives of the compounds of the general formulae I, XI, XII, XIII, XIV or XV, which are readily convertible in vivo into the required compound of the formulae I, XI, XII, XIII, XIV or XV. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

Whenever mentioned in relation to the compounds of the formulae I, XI, XII, XIII, XIV or XV, the terms epilepsy and epilepsies embrace any of the epilepsies, epileptic syndromes and epileptic seizures referred to in International League Against Epilepsy: Proposal for revised clinical and electroencephalographic classification of epileptic seizures. Commission on Classification and Terminology of the International League Against Epilepsy. Epilepsia 1981 22: 489-501 and in International League Against Epilepsy: Proposal for revised classification of epilepsies and epileptic syndromes. Commission on Classification and Terminology of the International League Against Epilepsy. Epilepsia 1989 30(4): 389-399.

Whenever mentioned in relation to the compounds of the formulae I, XI, XII, XIII, XIV or XV, the term anxiety disorders embraces conditions and diseases related to panic attack, agoraphobia, panic disorder with agoraphobia, panic disorder without agoraphobia, agoraphobia without history of panic disorder, specific phobia, social is phobia, obsessive-compulsive disorder, posttraumatic stress disorder, acute stress disorders, generalized anxiety disorder, anxiety disorder due to general medical condition, substance-induced anxiety disorder, separation anxiety disorder, adjustment disorders and anxiety disorder not otherwise specified as defined by American Psychiatric Association Diagnostic and statistical manual of mental disorders, 4ed 1994: 110-113, 393-444 and 623-627.

Pharmaceutical Compositions

The compounds of this invention are generally utilized as the free base or as a pharmaceutically acceptable salt thereof. Representative examples are mentioned above.

If desired, the pharmaceutical composition of the invention may comprise the compound of formula I in combination with further pharmacologically active substances such as those described in the foregoing.

The compounds of the invention may be administered alone or in combination with pharmaceutically acceptable carriers or excipients, in either single or multiple doses.

The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

The pharmaceutical compositions of this invention or those which are manufactured in accordance with this invention may be administered by any suitable route, for example orally in the form of tablets, capsules, powders, syrups, etc., or parenterally in the form of solutions for injection. For preparing such compositions, methods well known in the art may be used, and any pharmaceutically acceptable carriers, diluents, excipients or other additives normally used in the art may be used.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typically doses are in the order of about half the dose employed for oral administration.

The compounds of this invention are generally utilized as the free substance or as a pharmaceutically acceptable salt thereof. One example is a base addition salt of a compound having the utility of a free acid. When a compound of the invention contains a free acid such salts may be prepared in a conventional manner by treating a solution or suspension of a free acid of the compound of the invention with a chemical equivalent of a pharmaceutically acceptable base. Representative examples are mentioned above.

For parenteral administration, solutions of the novel compounds of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions for injections may be prepared by dissolving the active ingredient and possible additives in a part of the solvent for injection, preferably sterile water, adjusting the solution to a desired volume, sterilising the solution and filling it in suitable ampules or vials. Any suitable additive conventionally used in the art may be added, such as tonicity agents, preservatives, antioxidants, etc.

Suitable pharmaceutical carriers include inert solid diluents or fillers, sterile aqueous solution and various organic solvents.

Examples of solid carriers are lactose, terra alba, sucrose, cyclodextrin, talc, agar, pectin, acacia, stearic acid and lower alkyl ethers of cellulose corn starch, potato starch, talcum, magnesium stearate, gelatine, lactose, gums, and the like.

Any other adjuvants or additives usually used for such purposes such as colourings, flavourings, preservatives etc. may be used provided that they are compatible with the active ingredients.

Examples of liquid carriers are syrup, peanut oil, olive oil, phospho lipids, fatty acids, fatty acid amines, polyoxyethylene and water. Similarly, the carrier or diluent may include any sustained release material known in the art, such as glyceryl monostearate or glyceryl distearate, alone or mixed with a wax.

The pharmaceutical compositions formed by combining the novel compounds of the invention and the pharmaceutical acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include one or more suitable excipients. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablette, placed in a hard gelatine capsule in powder or pellet form or it can be in the form of a troche or lozenge.

The amount of solid carrier will vary widely but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

If desired, the pharmaceutical composition of the invention may comprise the compound of the formulae I, XI, XII, XIII, XIV or XV in combination with further pharmacologically active substances such as those described in the foregoing. Typical examples of recipes for the formulation of the invention are as follows:

1) Tablets containing 5.0 mg of a compound of the invention calculated as the free base:

| | |
|---|---|
| Compound of formulae I, XI, XII, XIII, XIV or XV | 5.0 mg |

| | |
|---|---|
| Lactose | 60 mg |
| Maize starch | 30 mg |
| Hydroxypropylcellulose | 2.4 mg |
| Microcrystalline cellulose | 19.2 mg |
| Croscarmellose Sodium Type A | 2.4 mg |
| Magnesium stearate | 0.84 mg |

2) Tablets containing 0.5 mg of a compound of the invention calculated as the free base:

| | |
|---|---|
| Compound of formulae I, XI, XII, XIII, XIV or XV | 0.5 mg |

| | |
|---|---|
| Lactose | 46.9 mg |
| Maize starch | 23.5 mg |
| Povidone | 1.8 mg |
| Microcrystalline cellulose | 14.4 mg |
| Croscarmellose Sodium Type A | 1.8 mg |
| Magnesium stearate | 0.63 mg |

3) Syrup containing per millilitre:

| | |
|---|---|
| Compound of formulae I, XI, XII, XIII, XIV or XV | 25 mg |

| | |
|---|---|
| Sorbitol | 500 mg |
| Hydroxypropylcellulose | 15 mg |
| Glycerol | 50 mg |
| Methyl-paraben | 1 mg |
| Propyl-paraben | 0.1 mg |
| Ethanol | 0.005 mL |
| Flavour | 0.05 mg |
| Saccharin sodium | 0.5 mg |
| Water | ad 1 mL |

4) Solution for injection containing per millilitre:

| | |
|---|---|
| Compound of formulae I, XI, XII, XIII, XIV or XV | 0.5 mg |

| | |
|---|---|
| Sorbitol | 5.1 mg |
| Acetic Acid | 0.05 mg |
| Saccharin sodium | 0.5 mg |
| Water | ad 1 ml |

Preparation of the Compounds of the Invention

The compounds of the invention of the general formula I, wherein R¹, R^{1′}, R², R^{2′}, R³, U, Y, X, Z, q and s are as defined above may be prepared by the methods as represented in the schemes and as described below:

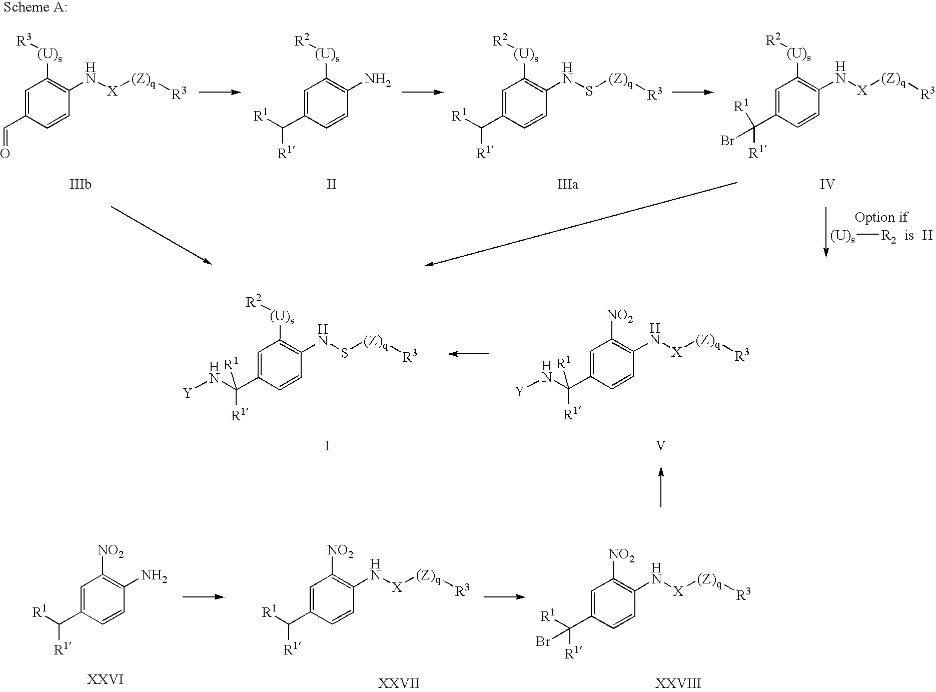

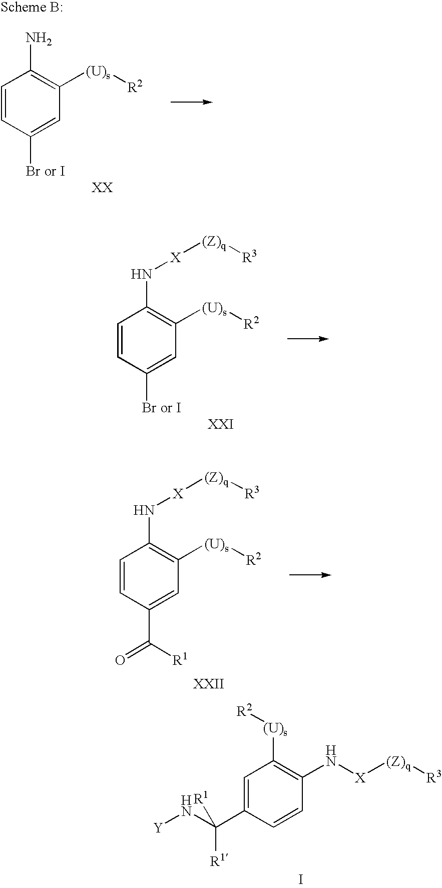

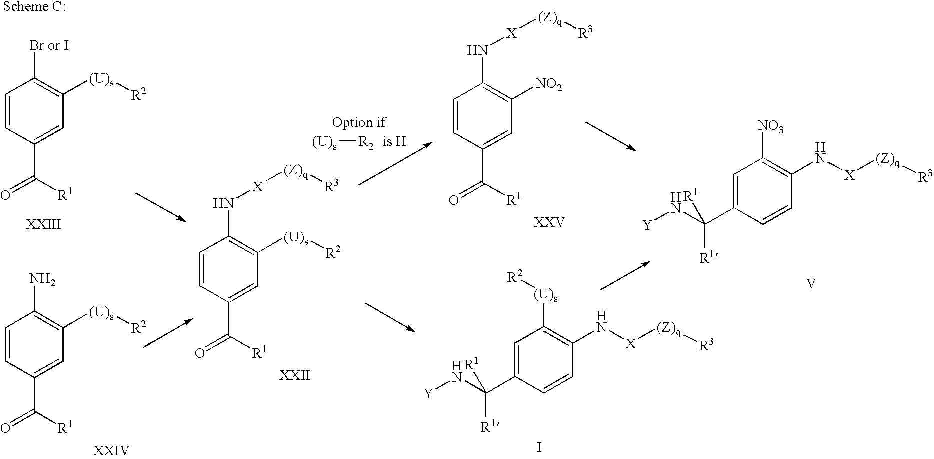

In the compounds of general formulae II, IIIa, IIIb, IV, V, XX, XXI, XXII, XXIII, XXIV, XXV, XXVI, XXVII and XXVIII R¹, R^{1′}, R², R^{2′}, R³, U, Y, X, Z, q and s are as defined under formula I, with the proviso that Rₗ is not acyl in compounds of general formulae XXII, XXIII, XXIV and XXV.

Compounds of the general formulae II, XX, XXIII, XXIV and XXVI are either obtained from commercial sources, or prepared by standard methods known to chemists skilled in the art.

Compounds of the general formulae IIIa and IIIb are either obtained from commercial sources, or prepared by standard methods known to chemists skilled in the art as outlined below.

Compounds of the general formula XXII, may be prepared by reacting suitably substituted p-bromo-anilines or p-iodo-anilines of general formula XX with suitable electrophilic reagents, such as suitably substituted acid chlorides, acid bromides, acid iodides, sulfonyl chlorides, carbamoyl chlorides, chloro formates, isocyanates and with or without the addition of bases, such as pyridine, trialkyl amines, potassium carbonate, magnesium oxide or lithium-, sodium-, or potassium alcoholates, in a suitable solvent, such as ethyl acetate, dioxane, tetrahydrofuran, acetonitrile, or diethyl ether, at a suitable temperature, such as room temperature or reflux temperature. These derivatives of general formula XXI are then subjected to an acid-base reaction at the NH-function using an alkylmagnesium halide, such as methylmagnesium halide, isopropylmagnesium halide, or using a dialkylmagnesium, such as dibutylmagnesium, as the base, in a suitable solvent such as tetrahydrofuran or diethylether at a suitable temperatutre such as −30° C. or 0° C. The acid-base reaction is then in situ followed by a lithium-halogen exchange by the addition of an alkyllithium such as n-butyllithium, sec-butyllithium or tert-butyllithium in a suitable solvent such as tetrahydrofuran or diethylether at a suitable temperatutre such as −78° C. or 0° C. The organometallic species are then reacted with a suitable formylating reagent such as DMF or a suitable acylating reagent such as acetylchloride. For general references see Wakefield (B. J. Wakefield, “Organomagnesium Methods in Organic Synthesis”, Academic Press, 1995) and Brandsma & Verkruijsse (L. Brandsma and H. D. Verkruijsse, “Preparative Polar Organometallic Chemistry”, Springer-Verlag, 1987).

Alternatively, compounds of the general formula XXII may be prepared by palladium catalysed C—N bond-forming reaction between suitably substituted p-bromo or p-iodo derivatives of general formula XXIII and suitably substituted amides or carbamates, as described by S. L. Buchwald et al. (J. Yin and S. L. Buchwald, Organic Letters, 2000, 2, 1101).

Alternatively, compounds of the general formula XXII may be prepared by reacting suitably substituted anilines of general formula XXIV with suitable electrophilic reagents, such as suitably substituted acid chlorides, acid bromides, acid iodides, sulfonyl chlorides, carbamoyl chlorides, chloro formates, isocyanates and with or without the addition of bases, such as pyridine, trialkyl amines, potassium carbonate, magnesium oxide or lithium-, sodium-, or potassium alcoholates, in a suitable solvent, such as ethyl acetate, dioxane, tetrahydrofuran, acetonitrile or diethyl ether, at a suitable temperature, such as room temperature or reflux temperature.

Compounds of general formula XXV may be prepared by nitration of compounds of formula XXII, in which (U)ₛ—R² is hydrogen, under standard nitrating conditions, such as acetic acid anhydride/nitric acid, sulphuric acid/nitric acid, sulfuric acid/sodium or potassium nitrate, trifluoroacetic acid/sodium or potassium nitrate at a suitable temperature such as 0° C. or room temperature.

Compounds of general formula XXVII may be prepared by reacting compounds of general formula XXVI with suitable electrophilic reagents, such as suitably substituted acid chlorides, acid bromides, acid iodides, sulfonyl chlorides, carbamoyl chlorides, chloro formates, isocyanates and with or without the addition of bases, such as pyridine, trialkyl amines, potassium carbonate, magnesium oxide or lithium-, sodium-, or potassium alcoholates, in a suitable solvent, such as ethyl acetate, dioxane, tetrahydrofuran, acetonitrile or diethyl ether, at a suitable temperature, such as room temperature or reflux temperature.

Compounds of general formula XXVIII may be prepared from compounds of the general formula XXVII by radical halogenation reactions known to the chemist skilled in the art, such as reaction with N-bromosuccinimide and dibenzoylperoxide, in a suitable solvent, such as tetrachloromethane or benzene at a suitable temperature, such as reflux temperature.

Some compounds of general formula V may be prepared by reductive amination reactions of compounds of the general formula XXV, known to the chemist skilled in the art, with amines of type Y—NH₂, using reducing agents, such as sodium borohydride or sodium cyanoborohydride in a suitable solvent, such as methanol, ethanol, tetrahydrofuran, water, dioxane or mixtures thereof, with or without addition of catalytic amounts of acid, such as acetic acid or hydrochloric acid, at a suitable temperature.

Alternatively, compounds of general formula XXV can be reacted with amines of type Y—NH₂, in a suitable solvent, such as methanol, ethanol, tetrahydrofuran, dioxane, xylene or mixtures thereof, with or without addition of catalytic amounts of acid, such as acetic acid, at a suitable temperature, to form imines, that can be isolated by crystallisation or by evaporation of the solvent. The imines can then be reduced using reducing agents, such as sodium borohydride or sodium cyanoborohydride in a suitable solvent, such as methanol, ethanol, tetrahydrofuran, water, dioxane or mixtures thereof, with or without addition of catalytic amounts of acid, such as acetic acid, at a suitable temperature, to give compounds of the general formula V.

Alternatively, some compounds of the general formula V may be prepared by the reaction of compounds of the general formula XXVIII with amines of type Y—NH₂, for example 4-tert-butylaniline, in a suitable solvent, such as tetrahydrofuran, dioxane or N,N-dimethylformamide, with or without addition of bases, such as trialkyl amines or potassium carbonate, at a suitable temperature.

Alternatively, some compounds of general formula V may be prepared by nitration of compounds of general structure IV, in which (U)ₛ—R² is hydrogen, under standard nitrating conditions, such as acetic acid anhydride/nitric acid, sulphuric acid/nitric acid, sulfuric acid/sodium or potassium nitrate, trifluoroacetic acid/sodium or potassium nitrate at a suitable temperature such as 0° C. or room temperature, followed by reaction with amines of type Y—NH₂, for example 4-tert-butylaniline, in a suitable solvent, such as tetrahydrofuran, dioxane or N,N-dimethylformamide, with or without addition of bases, such as trialkyl amines or potassium carbonate, at a suitable temperature.

Altenatively, appropriate carboxylic acids are reduced with appropriate reducing agents, such as borane, and carboxylic acid esters are reduced with appropriate reducing agents, such as diisobutyl aluminium hydride. The resulting benzylic alcohols are then reacted with a suitable oxidant, such as tetrapropylammonium perruthenate/N-methylmorpholin-N-oxide, pyridinium chlorochromat or dimethylsulfoxide/oxalylchloride, to give compounds of general formula IIIb. Additionally, for further variation of R², compounds of the general formula IIIa, wherein R²=methyl, U=oxygen, and s=1, can be demethylated by methods known to chemists skilled in the art, such as treatment with boron tribromide in a suitable solvent, such as dichloromethane, at a suitable temperature, such as 0° C. or room temperature. The resulting phenols can then be transformed into compounds of the general formula IIIa, wherein U=oxygen, and s=1, by methods known to chemists skilled in the art. Such methods include: (a) the reaction with electrophiles, such as alkyl chlorides, alkyl bromides, alkyl iodides, benzyl chlorides, benzyl bromides, benzyl iodides, carbonic acid chlorides, carbonic acid bromides, or carbonic acid anhydrides in the presence of suitable bases, such as potassium carbonate, in a suitable solvent, such as tetrahydrofuran, N,N-dimethylformamide, or 1,2-dichloroethane, at suitable temperatures, such as room temperature or reflux temperature; (b) the reaction with alkyl, benzylic, or heteroarylalkyl alkohols under conditions known as the Mitsunobu reaction (O. Mitsunobu Synthesis 1981, 1).

Alternatively, compounds of the general formula IIIa may be prepared by the reaction of compounds of the general structure II with suitable electrophilic reagents, such as suitably substituted acid chlorides, acid bromides, acid iodides, sulfonyl chlorides, carbamoyl chlorides, chloro formates, isocyanates and with or without the addition of bases, such as pyridine, trialkyl amines, potassium carbonate, magnesium oxide or lithium-, sodium-, or potassium alcoholates, in a suitable solvent, such as ethyl acetate, dioxane, tetrahydrofuran, acetonitrile or diethyl ether, at a suitable temperature, such as room temperature or reflux temperature.

Compounds of the general formula IV may be prepared from compounds of the general formula IIIa by radical halogenation reactions known to the chemist skilled in the art, such as reaction with N-bromosuccinimide and dibenzoylperoxide, in a suitable solvent, such as tetrachloromethane or benzene at a suitable temperature, such as reflux temperature.

Some compounds of the general formula I may be prepared by the reaction of compounds of the general formula IV with amines of type Y—NH₂, for example 4-tert-butylaniline, in a suitable solvent, such as tetrahydrofuran, dioxane or N,N-dimethylformamide, with or without addition of bases, such as trialkyl amines or potassium carbonate, at a suitable temperature.

Compounds of the general formula I can be prepared by reductive amination reactions of compounds of the general formulae IIIb and XXII, known to the chemist skilled in the art, with amines of type Y—NH₂, using reducing agents, such as sodium borohydride or sodium cyanoborohydride in a suitable solvent, such as methanol, ethanol, tetrahydrofuran, water, dioxane or mixtures thereof, with or without addition of catalytic amounts of acid, such as acetic acid or hydrochloric acid, at a suitable temperature.

Alternatively, compounds of the general formulae IIIb and XXII can be reacted with amines of type Y—NH₂, in a suitable solvent, such as methanol, ethanol, tetrahydrofuran, dioxane, xylene or mixtures thereof, with or without addition of catalytic amounts of acid, such as acetic acid, at a suitable temperature, to form imines, that can be isolated by crystallisation or by evaporation of the solvent. The imines can then be reduced using reducing agents, such as sodium borohydride or sodium cyanoborohydride in a suitable solvent, such as methanol, ethanol, tetrahydrofuran, water, dioxane or mixtures thereof, with or without addition of catalytic amounts of acid, such as acetic acid, at a suitable temperature, to give compounds of the general formula I.

Alternatively, compounds of the general formula I may be prepared by the reaction of compounds of the general formula V with a suitable reducing agent, such as iron or zinc powder in aqueous hydrochloric acid or in alcoholic ammonium chloride or aqueous sodium dithionite, in a suitable solvent, such as tetrahydrofuran or ethanol.

To obtain compounds of the general formula I, where U is NR^{2′} and s is 1 and R² and optionally R^{2′} are not hydrogen, a protecting group, such as tertbutyloxycarbonyl, is introduced at the benzylic nitrogen in compounds of the general formula V, before the reduction of the nitro group, by methods known to the chemist skilled in the art. This protecting group is cleaved by known methods, after the introduction of R² and optionally R^{2′}, said introduction is accomplished by using one or more of the following methods:

Introduction of R² by a reductive alkylation procedure using suitable aldehydes and reducing agents, such as sodium borohydride or sodium cyanoborohydride in a suitable solvent, such as methanol, ethanol, tetrahydrofuran, water, dioxane or mixtures thereof, with or without addition of catalytic amounts of acid, such as acetic acid, at a suitable temperature, as described above.

Optional introduction of R^{2′} by an additional reductive alkylation procedure using suitable aldehydes and reducing agents, such as sodium borohydride or sodium cyanoborohydride in a suitable solvent, such as methanol, ethanol, tetrahydrofuran, water, dioxane or mixtures thereof, with or without addition of catalytic amounts of acid, such as acetic acid, at a suitable temperature, as described above.

Alternatively, R^{2′} or R² may be introduced by an acylation reaction using suitable electrophilic reagents, such as acid chlorides, acid bromides, acid iodides, sulfonyl chlorides, and alkyl formates with the addition of bases, such as trialkyl amines, potassium carbonate, magnesium oxide or lithium-, sodium-, or potassium alcoholates, in a suitable solvent, such as ethyl acetate, dioxane, tetrahydrofuran, acetonitrile or diethyl ether, at a suitable temperature, as described above.

### EXAMPLES

Analytical LC-MS data were obtained on a PE Sciex API 150EX instrument equipped with IonSpray source and Shimadzu LC-8A/SLC-10A LC system. Column: 30×4.6 mm Waters Symmmetry C18 column with 3.5 μm particle size; Solventsystem: A=water/trifluoroacetic acid (100:0.05) and B=water/acetonitrile/trifluoroacetic acid (5:95:0.03); Method: Linear gradient elution with 90% A to 100% B in 4 minutes and with a flow rate of 2 mL/min. Purity is expressed in % and was determined by integration of the UV (254 nm) and ELSD trace. The retention times (RT) are expressed in minutes.

¹H NMR spectra were recorded at 500.13 MHz and ¹³C NMR spectra were recorded at 125.76 MHz, both on a Bruker Avance DRX500 instrument. Deuterated chloroform (99.8% D) or dimethyl sulfoxide (99.8% D) were used as solvents. TMS was used as internal reference standard. Chemical shift values are expressed in ppm-values. The following abbreviations are used for multiplicity of NMR signals: s=singlet, d=doublet, t=triplet, q=quartet, qui=quintet, h=heptet, dd=double doublet, dt=double triplet, dq=double quartet, tt=triplet of triplets, m=multiplet and br=broad.

Preparation of Intermediates

### (4-Methyl-2-nitrophenyl)-carbamic acid ethyl ester

4-Methyl-2-nitroaniline (45.7 g, 0.30 mol) was dissolved in tetrahydrofuran (350 mL) and K₂CO₃ (50 g, 0.36 mol) was added. Ethyl chloroformate (39.1 g, 0.36 mol) dissolved in THF (50 mL) was added and the solution refluxed for 18 hours. The mixture was cooled to ambient temperature and the solid filtered off. The solution was concentrated in vacuo and the resulting solid recrystallised from ethanol to give 47.1 g (70%) of the title compound as yellow crystals. LC/MS (m/z) 224.9 (MH⁺); RT=3.08. ¹H NMR (CDCl₃): 1.35 (t, 3H); 2.38 (s, 3H); 4.28 (q, 2H); 7.42 (d, 1H); 8.00 (s, 1H); 8.45 (d, 1H); 9.70 (s, 1H). ¹³C NMR (CDCl₃): 14.4, 20.4, 61.9, 120.7, 125.6, 132.4, 133.1, 135.9, 136.9, 153.3.

The following intermediates were prepared analogously:

### (4-Bromo-2-chlorophenyl)carbamic acid ethyl ester

LC/MS (m/z) 278.9 (M⁺); RT=3.45. ¹H NMR (CDCl₃): 1.38 (t, 3H); 4.30 (q, 2H); 7.10 (br s, 1H); 7.42 (d, 1H); 7.55 (s, 1H); 8.12 (d, 1H). ¹³C NMR (CDCl₃): 14.9, 62.2, 115.5, 121.3, 123.0, 131.2, 131.8, 134.5, 153.4.

### (4-Bromo-2-fluorophenyl)carbamic acid ethyl ester

LC/MS (m/z) 262.8 (M⁺); RT=3.21. ¹H NMR (CDCl₃): 1.35 (t, 3H); 4.27 (q, 2H); 6.80 (br s, 1H); 7.30 (br m, 2H); 8.05 (br m, 1H).

### (4-Bromo-2-methylphenyl)carbamic acid ethyl ester

LC/MS (m/z) 258.6 (M⁺); RT=3.67. ¹H NMR (CDCl₃): 1.38 (t, 3H); 2.35 (s, 3H); 4.30 (q, 2H); 7.05 (br s, 1H); 7.50 (d, 1H); 7.65 (s, 1H); 8.20 (d, 1H).

### (4-Acetylphenyl)carbamic acid ethyl ester

LC/MS (m/z) 207.8 (M⁺); RT=2.16. ¹H NMR (CDCl₃): 1.35 (t, 3H); 2.60 (s, 3H); 4.27 (q, 2H); 7.00 (br s, 1H); 7.50 (d, 2H); 7.95 (d, 2H). ¹³C NMR (CDCl₃): 14.9, 26.8, 62.0, 118.0, 130.3, 132.5, 142.9, 153.5, 197.4.

### N-(4-bromo-2-chlorophenyl)butyramide

LC/MS (m/z) 277.8 (M⁺); RT=3.00. ¹H NMR (CDCl₃): 0.95 (t, 3H); 1.70 (m, 2H); 2.35 (t, 2H); 7.32 (d, 1H); 7.45 (s, 1H); 7.50 (br s, 1H); 8.25 (d, 1H). ¹³C NMR (CDCl₃): 14.1, 19.8, 40.3, 117.1, 122.9, 123.5, 131.2, 131.8, 134.3, 171.6.

### N-(4-bromophenyl)butyramide

¹H NMR (CDCl₃): 1.05 (t, 3H); 1.80 (m, 2H); 2.35 (t, 2H); 7.15 (br s, 1H); 7.45 (br m, 4H).

### N-(4-bromo-2-fluorophenyl)butyramide

¹H NMR (CDCl₃): 1.05 (t, 3H); 1.75 (m, 2H); 2.45 (t, 2H); 7.30 (br m, 3H); 8.30 (br m, 1H).

### N-(4-bromo-2-methylphenyl)butyramide

LC/MS (m/z) 256.0 (M⁺); RT=2.60. ¹H NMR (CDCl₃): 1.05 (t, 3H); 1.80 (m, 2H); 2.25 (s, 3H); 2.40 (t, 2H); 6.90 (br s, 1H); 7.35 (br m, 2H); 7.80 (br d, 1H). ¹³C NMR (CDCl₃): 13.8, 17.6, 19.0, 39.5, 180.0, 124.9, 127.0, 130.5, 132.7, 133.1, 134.8, 171.3.

### (4-Bromomethyl-2-nitrophenyl)-carbamic acid ethyl ester

(4-Methyl-2-nitrophenyl)-carbamic acid ethyl ester (22.4 g, 0.10 mol) is dissolved in CCl₄ (300 mL) and N-bromosuccinimide (17.8 g, 0.10 mmol) is added followed by dibenzoylperoxid (0.73 g, 0.003 mol). The mixture is stirred at reflux for 12 hours and then cooled to ambient temperature. The mixture was filtered and the solution concentrated in vacuo. The remaining solid was recrystallised from methanol to give 17.0 g (56%) of the title compound as bright yellow crystals. LC/MS (m/z) 304.3 (MH⁺); RT=3.22. ¹H NMR (CDCl₃): 1.38 (t, 3H); 4.29 (q, 2H); 4.50 (s, 2H); 7.65 (d, 1H); 8.25 (s, 1H); 8.58 (d, 1H); 9.85 (s, 1H). ¹³C NMR (CDCl₃): 14.8, 31.5, 62.5, 121.6, 126.5, 132.4, 135.9, 136.0, 136.8, 153.4.

### (2-Chloro-4-formylphenyl)carbamic acid ethyl ester

To (4-Bromo-2-chlorophenyl)carbamic acid ethyl ester (6.1 g, 21.9 mmol) dissolved in dry tetrahydrofuran (100 mL), kept under an argon atmosphere and cooled on an ice bath, was added dibutylmagnesium (11 mL of a 1.0 M solution in heptane, 11 mmol) and the solution was stirred for 30 minutes. The solution was then cooled to −78° C. using an acetone/dry-ice bath and n-butyllithium (15 mL of a 1.6 M solution in hexane) was added over 30 minutes, while keeping the temperature below −70° C. The mixture was stirred for 1 hour, and then DMF (3.2 g, 43.8 mmol) was added dropwise. The mixture was allowed to reach ambient temperature, stirred for 1 hour, and quenched by addition of aqueous ammoniumchloride (50 mL). The phases were separated and the organic phase was evaporated in vacuo. The product was purified by column chromatography using silica gel on a Flashmaster system eluting with heptane/ethyl acetate (linear gradient from 1:0 to 5:1). Fractions containing the product were pooled and evaporation in vacuo gave the title compound as a white solid (2.5 g, 50%)

LC/MS (m/z) 228.1 (M⁺); RT=2.55. ¹H NMR (CDCl₃): 1.30 (t, 3H); 4.22 (q, 2H); 7.45 (br s, 1H); 7.70 (d, 1H); 7.85 (s, 1H); 8.38 (d, 1H); 9.80 (s, 1H). ¹³C NMR (CDCl₃): 14.4, 62.2, 118.9, 122.2, 129.9, 130.2, 131.7, 140.2, 152.7, 189.7.

The following intermediates were prepared analogously:

### (2-Fluoro-4-formylphenyl)carbamic acid ethyl ester

LC/MS (m/z) 211.9 (M⁺); RT=2.24. ¹H NMR (CDCl₃): 1.38 (t, 3H); 4.32 (q, 2H); 7.15 (br s, 1H); 7.60-7.72 (br m, 2H); 8.40 (br m, 1H); 9.90 (s, 1H). ¹³C NMR (CDCl₃): 14.4, 62.1, 114.2, 119.1, 128.2, 131.5, 132.6, 150.7, 152.7, 190.0.

### (4-Formyl-2-methylphenyl)carbamic acid ethyl ester

LC/MS (m/z) 207.8 (M⁺); RT=2.18. ¹H NMR (CDCl₃): 1.38 (t, 3H); 2.40 (s, 3H); 4.30 (q, 2H); 6.70 (br s, 1H); 7.70 (d, 1H); 7.80 (s, 1H); 8.25 (d, 1H); 9.93 (s, 1H). ¹³C NMR (CDCl₃): 14.5, 17.5, 61.8, 118.6, 125.7, 130.1, 131.2, 131.4, 142.0, 153.1, 191.3.

### N-(4-formylphenyl)butyramide

LC/MS (m/z) 192.0 (M⁺); RT=1.94. ¹H NMR (CDCl₃): 1.00 (t, 3H); 1.80 (m, 2H); 2.40 (t, 2H); 7.60 (br s, 1H); 7.72 (d, 2H); 7.85 (d, 2H); 9.95 (s, 1H). ¹³C NMR (CDCl₃): 13.7, 18.9, 39.8, 119.2, 131.2, 132.2, 143.6, 171.7, 191.1.

### N-(2-chloro-4-formylphenyl)butyramide

LC/MS (m/z) 225.7 (M⁺); RT=2.39. ¹H NMR (CDCl₃): 1.08 (t, 3H); 1.82 (m, 2H); 2.50 (t, 2H); 7.80 (d, 1H); 7.90 (br s, 1H); 7.95 (s, 1H); 8.70 (d, 1H); 9.90 (s, 1H). ¹³C NMR (CDCl₃): 13.7, 18.8, 40.0, 120.6, 122.8, 129.6, 130.3, 132.2, 139.8, 171.5, 189.8.

### N-(2-fluoro-4-formylphenyl)butyramide

LC/MS (m/z) 210.0 (M⁺); RT=2.10. ¹H NMR (CDCl₃): 1.05 (t, 3H); 1.80 (m, 2H); 2.45 (t, 2H); 7.60 (m, 1H); 7.70 (br m, 3H); 8.65 (m, 1H); 9.90 (s, 1H). ¹³C NMR (CDCl₃): 13.7, 18.8, 39.8, 113.9, 120.8, 128.3, 132.2, 151.0, 152.9, 171.7, 190.0.

### N-(4-formyl-2-methylphenyl)butyramide

LC/MS (m/z) 206.0 (M⁺); RT=1.98. ¹H NMR (CDCl₃): 1.05 (t, 3H); 1.85 (m, 2H); 2.35 (s, 3H); 2.45 (t, 2H); 7.10 (br s, 1H); 7.70 (br m, 2H); 7.80 (br m, 1H); 8.35 (br m, 1H); 9.95 (s, 1H). ¹³C NMR (CDCl₃): 13.7, 17.6, 19.0, 39.8, 121.1, 127.0, 129.9, 131.2, 132.2, 141.7, 171.3, 191.3.

### (4-Formylphenyl)carbamic acid ethyl ester

4-Bromobenzaldehyde (9.25 g, 50 mmol), ethyl carbamate (5.35 g, 60 mmol), bis(dibenzylideneacetone)palladium (288 mg, 1.0 mol %), 4,5-bis(diphenylphosphino)-9,9-dimethylxanthene (435 mg, 1.5 mol %) and cesiumcarbonate (23.0 g, 70 mmol) was suspended in dry tetrahydrofuran (100 mL) under an argon atmosphere and the mixture was heated to 80° C. over night. After cooling to ambient temperature the mixture was diluted with ethyl acetate (100 mL), filtered and purified by filtration through a short plug of silica gel. Evaporation in vacuo gave the title compound as a yellow solid (9.3 g, 96.3%).

LC/MS (m/z) 193.7 (M⁺); RT=2.12. ¹H NMR (CDCl₃): 1.28 (t, 3H); 4.20 (q, 2H); 6.95 (br s, 1H); 7.50 (d, 2H); 7.78 (d, 2H); 9.85 (s, 1H). ³C NMR (CDCl₃): 14.9, 62.2, 118.4, 131.7, 132.0, 144.2, 153.4, 191.4.

The following intermediate was prepared analogously:

### (4-Formyl-2-nitrophenyl)carbamic acid tert-butyl ester

¹H NMR (CDCl₃): 1.55 (s, 9H); 8.12 (d, 1H); 8.70 (s, 1H); 8.82 (d, 1H); 9.90 (s, 1H); 10.05 (br s, 1H).

### (4-Formyl-2-nitrophenyl)carbamic acid ethyl ester

(4-Formylphenyl)carbamic acid ethyl ester (6.34 g, 32.8 mmol) was disolved in concentrated sulphuric acid (150 mL) cooled to 0° C. using an ice bath. Sodium nitrate (2.92 g, 34.4 mmol) was added in small portions over 20 minutes. After complete addition the mixture was stirred 3 hours at 0° C. and then poured onto chrushed ice. The yellow precipitate was filtered off, washed thoroughly with water and dried in vacuo to give 7.11 g (91%) of the title compound.

LC/MS (m/z) 238.5 (M⁺); RT=2.62. ¹H NMR (CDCl₃): 1.40 (t, 3H); 4.32 (q, 2H); 8.15 (d, 1H); 8.75 (s, 1H); 8.85 (d, 1H); 9.95 (s, 1H); 10.15 (br s, 1H). ¹³C NMR (CDCl₃): 14.7, 63.0, 121.2, 128.9, 130.5, 131.7, 135.6, 140.7, 153.0, 189.2.

The following intermediate was prepared analogously:

### (4-Acetyl-2-nitrophenyl)carbamic acid ethyl ester

LC/MS (m/z) 252.8 (M⁺); RT=2.69. ¹H NMR (CDCl₃): 1.40 (t, 3H); 2.65 (s, 3H); 4.35 (q, 2H); 8.22 (d, 1H); 8.75 (d, 1H); 8.85 (s, 1H); 10.10 (br s, 1H). ¹³C NMR (CDCl₃): 14.7, 26.7, 62.9, 120.8, 127.0, 131.4, 135.4, 135.6, 139.6, 153.3, 195.2.

### (4′-Dimethylamino-5-formylbiphenyl-2-yl)carbamic acid ethyl ester

(2-Bromo-4-formylphenyl)carbamic acid ethyl ester (0.50 g, 1.84 mmol), 4-dimethylamino-phenyl-boronic acid (0.90 g, 5.52 mmol) and palladium(II)acetate (0.040 g, 0.18 mmol) was suspended in acetone (15 mL) and 5 M potassium carbonate (2 mL, 10 mmol) was added. The mixture was heated to 125° C. for 10 minutes in a microwave oven. After cooling to ambient temperaure, the mixture was filtered, concentrated in vacuo and purified by column chromatography using silica gel on a Flashmaster system eluting with heptane/ethyl acetate (linear gradient from 1:0 to 3:1). Fractions containing the product were pooled and evaporation in vacuo gave the title compound as a yellow solid (260 mg, 45%).

¹H NMR (CDCl₃): 1.25 (t, 3H); 3.05 (s, 6H); 4.20 (q, 2H); 6.82 (d, 2H); 7.10 (br s, 1H); 7.21 (d, 2H); 7.70 (s, 1H); 7.82 (d, 1H); 8.40 (d, 1H); 9.95 (s, 1H).

The following intermediate was prepared analogously:

### (4′-Chloro-5-formylbiphenyl-2-yl)carbamic acid ethyl ester

¹H NMR (CDCl₃): 1.28 (t, 3H); 4.20 (q, 2H); 6.78 (br s, 1H); 7.30 (d, 2H); 7.50 (d, 2H); 7.70 (s, 1H); 7.88 (d, 1H); 8.45 (d, 1H); 9.95 (s, 1H).

### {4-[(4-tert-Butylphenylamino)-methyl]-2-nitrophenyl}-carbamic acid ethyl ester

(4-Bromomethyl-2-nitrophenyl)-carbamic acid ethyl ester (0.5 g, 1.65 mmol), 4-tert-butyl-aniline (0.28 g, 1.8 mmol) and K₂CO₃ (0.35 g, 2.5 mmol) were mixed in tetrahydrofuran (15 mL) and heated to reflux temperature for 12 hours. The mixture was cooled to ambient temperature, filtered and evaporated to dryness in vacuo. Purified by chromatography using silica gel on a Flashmaster system eluting with heptane/ethyl acetate (linear gradient from 1:0 to 5:1). Fractions containing the product were pooled and evaporated in vacuo to yield the title compound as a yellow solid (400 mg, 65%).

LC/MS (m/z) 372.2 (MH⁺); RT=3.58, WV purity=97.9, ELS purity=98.1.

The following intermediates were prepared analogously:

### (2-Nitro-4-phenylaminomethyphenyl)-carbamic acid ethyl ester

LC/MS (m/z) 315.0 (M⁺); RT=3.12, W purity=92.1, ELS purity=95.0.

### {2-Nitro-4-[(4-trifluoromethylphenylamino)-methyl]-phenyl}-carbamic acid ethyl ester

LC/MS (m/z) 383.1 (MH⁺); RT=3.62, WV purity=86.0, ELS purity=98.2.

### {4-[(4-Chlorophenylamino)-methyl]-2-nitrophenyl}-carbamic acid ethyl ester

LC/MS (m/z) 349.1 (M⁺); RT=3.58, UV purity=96.0, ELS purity=98.7.

### [4-(Naphthalen-2-ylaminomethyl)-2-nitrophenyl]-carbamic acid ethyl ester

LC/MS (m/z) 366.3 (MH⁺); RT=3.62, UV purity=87.9, ELS purity=92.3.

### [2-Nitro-4-(p-tolylamino-methyl)-phenyl]-carbamic acid ethyl ester

LC/MS (m/z) 330.1 (MH⁺); RT=2.87, WV purity=97.1, ELS purity=98.4.

### {4-[(3-Fluorophenylamino)-methyl]-2-nitrophenyl}-carbamic acid ethyl ester

LC/MS (m/z) 332.0 (M⁺); RT=3.33, UV purity=84.8, ELS purity=96.1.

### {4-[(4-Fluorophenylamino)-methyl]-2-nitrophenyl}-carbamic acid ethyl ester

LC/MS (m/z) 332.0 (M⁺); RT=3.10, UV purity=98.2, ELS purity=98.8. ¹H NMR (CDCl₃): 1.33 (t, 3H); 4.00 (br s, 1H, NH); 4.28 (q, 2H); 4.33 (s, 2H); 6.52 (m, 2H); 6.88 (m, 2H); 7.65 (m, 1H); 8.20 (s, 1H); 8.55 (m, 1H); 9.78 (s, 1H, NH).

### {4-[(2-Fluorophenylamino)-methyl]-2-nitrophenyl}-carbamic acid ethyl ester

LC/MS (m/z) 333.8 (MH⁺); RT=3.41, UV purity=93.8, ELS purity=96.1.

### [4-(Biphenyl-4-ylaminomethyl)-2-nitrophenyl]-carbamic acid ethyl ester

LC/MS (m/z) 392.3 (MH⁺); RT=3.74, UV purity=87.0, ELS purity=94.5.

### {4-[(2,4-Difluorophenylamino)-methyl]-2-nitrophenyl}-carbamic acid ethyl ester

LC/MS (m/z) 351.3 (M⁺); RT=3.45, UV purity=96.1, ELS purity=97.2.

### {4-[(4-Methoxyphenylamino)-methyl]-2-nitrophenyl}-carbamic acid ethyl ester

LC/NS (m/z) 346.1 (MH⁺); RT=2.16, UV purity=87.3, ELS purity=96.9.

### {4-[(4-Cyclohexylphenylamino)-methyl]-2-nitrophenyl}-carbamic acid ethyl ester

LC/MS (m/z) 397.2 (M⁺); RT=3.87, UV purity=95.8, ELS purity=98.6.

### [4-(Indan-5-ylaminomethyl)-2-nitrophenyl]-carbamic acid ethyl ester

LC/MS (m/z) 355.2 (M⁺); RT=2.97, UV purity=97.1, ELS purity=99.2.

### {4-[(4-Isopropylphenylamino)-methyl]-2-nitrophenyl}-carbamic acid ethyl ester

LC/MS (m/z) 358.1 (MH⁺); RT=3.33, UV purity=98.1, ELS purity=99.3.

### {4-[(4-Butylphenylamino)-methyl]-2-nitrophenyl}-carbamic acid ethyl ester

LC/MS (m/z) 371.8 (M⁺); RT=3.10, UV purity=92.3, ELS purity=94.1.

### {4-[2-(4-Chloro-3-fluorophenyl)ethyl]-2-nitrophenyl}carbamic acid ethyl ester

A solution of (4-formyl-2-nitrophenyl)carbamic acid ethyl ester (1.40 g, 5.88 mmol) and 4-chloro-3-fluoroaniline (0.86 g, 5.88 mmol) in dry toluene (30 mL) was refluxed for 12 hours, and the toluene was removed in vacuo. The solids were then redissolved in ethanol (30 mL) and acetic acid (3 mL) and treated with sodium cyanoborohydride (1.50 g, 23.8 mmol). After stirring for 1 hour at ambient temperature the mixture was treated with a second batch of sodium borohydride (1.50 g, 23.8 mmol) and stirred for 3 hours. The reaction was quenched with aqueos sodium hydrogencarbonate (50 mL) and the precipitate filtered off, washed with water and dried in vacuo to give the title compound as a yellow solid (1.84 g, 85%).

LC/MS (m/z) 367.1 (M⁺); RT=3.70, WV purity=94.2, ELS purity=97.7. ¹H NMR (CDCl₃): 1.38 (t, 3H); 4.15 (q, 2H); 4.28 (d, 2H); 4.65 (br, 1H); 6.28 (m, 2H); 7.05 (m, 1H); 7.55 (d, 1H); 8.10 (s, 1H); 8.50 (d, 1H); 9.70 (s, 1H).

The following intermediates were prepared analogously:

### {4-[(2,4-Dichlorophenylamino)methyl]-2-nitrophenyl}carbamic acid ethyl ester

¹H NMR (CDCl₃): 1.35 (t, 3H); 4.25 (q, 2H); 4.40 (d, 2H); 4.82 (br t, 1H); 6.45 (d, 1H); 7.02 (d, 1H); 7.22 (m, 1H); 7.60 (d, 1H); 8.15 (m, 1H); 8.55 (d, 1H); 9.75 (br s, 1H).

### {4-[(2,3-Dichlorophenylamino)methyl]-2-nitrophenyl}carbamic acid ethyl ester

¹H NMR (CDCl₃): 1.33 (t, 3H); 4.25 (q, 2H); 4.45 (d, 2H); 5.00 (br t, 1H); 6.40 (d, 1H); 6.80 (d, 1H); 6.98 (m, 1H); 7.60 (m, 1H); 8.17 (m, 1H); 8.55 (d, 1H); 9.70 (br s, 1H).

### {4-[(3,5-Dichlorophenylamino)methyl]-2-nitrophenyl}carbamic acid ethyl ester

¹H NMR (CDCl₃): 1.35 (t, 3H); 4.20-4.40 (br m, 5H); 6.45 (s, 2H); 6.70 (m, 1H); 7.60 (d, 1H); 8.15 (m, 1H); 8.58 (d, 1H); 9.80 (br s, 1H).

### {4-[(3,4-Dichlorophenylamino)methyl]-2-nitrophenyl}carbamic acid ethyl ester

¹H NMR (CDCl₃): 1.33 (t, 3H); 4.22-4.40 (m, 5H); 6.42 (dd, 1H); 6.65 (d, 1H); 7.20 (d, 1H); 7.58 (d, 1H); 8.15 (s, 1H); 8.55 (d, 1H); 9.80 (br s, 1H).

### {2-Nitro-4-[(3-trifluoromethylphenylamino)methyl]phenyl}carbamic acid ethyl ester

¹H NMR (CDCl₃): 1.35 (t, 3H); 4.27 (q, 2H); 4.40 (br m, 3H); 6.70 (d, 1H); 6.82 (br s, 1H); 6.98 (d, 1H); 7.22 (m, 1H); 7.62 (d, 1H); 8.20 (s, 1H); 8.55 (d, 1H); 9.75 (br s, 1H).

### {4-[(3-Fluoro-4-trifluoromethylphenylamino)methyl]-2-nitrophenyl}carbamic acid ethyl ester

LC/MS (m/z) 401.5 (M⁺); RT=3.74, UV purity=92.1, ELS purity=98.5. ¹H NMR (CDCl₃): 1.28 (t, 3H); 4.15 (q, 2H); 4.25 (d, 2H); 4.60 (br, 1H); 6.18-6.28 (br m, 2H); 7.32 (m, 1H); 7.50 (d, 1H); 8.05 (s, 1H); 8.45 (d, 1H); 9.70 (s, 1H).

### {4-[(3,4-Difluorophenylamino)methyl]-2-nitrophenyl}carbamic acid ethyl ester

LC/MS (m/z) 351.1 (M⁺); RT=3.53, UV purity=84.5, ELS purity=98.2. ¹H NMR (CDCl₃): 1.30 (t, 3H); 4.20 (br, 1H); 4.28 (q, 2H); 4.35 (s, 2H); 6.22 (m, 1H); 6.40 (m, 1H); 6.95 (m, 1H); 7.60 (d, 1H); 8.20 (s, 1H); 8.55 (d, 1H); 9.75 (s, 1H).

### {4-[(4-Cyanophenylamino)methyl]-2-nitrophenyl}carbamic acid ethyl ester

LC/MS (m/z) 340.5 (M⁺); RT=3.20, UV purity=85.6, ELS purity=93.1. ¹H NMR (CDCl₃): 1.35 (t, 3H); 4.05 (br, 1H); 4.25 (q, 2H); 4.40 (s, 2H); 6.60 (d, 2H); 7.40 (d, 2H); 7.55 (d, 1H); 8.15 (s, 1H); 8.55 (d, 1H); 9.77 (s, 1H).

### {4-[(4-Fluoro-3-trifluoromethylphenylamino)methyl]-2-nitrophenyl}carbamic acid ethyl ester

LC/MS (m/z) 401.0 (M⁺); RT=3.74, UV purity=92.7, ELS purity=99.6. ¹H NMR (CDCl₃): 1.35 (t, 3H); 4.00 (br, 1H); 4.25 (q, 2H); 4.35 (s, 2H); 6.65 (m, 1H); 6.75 (m, 1H); 7.00 (m, 1H); 7.60 (d, 1H); 8.15 (s, 1H); 8.55 (d, 1H); 9.80 (s, 1H).

### {4-[(3-Chloro-4-methylphenylamino)methyl]-2-nitrophenyl}carbamic acid ethyl ester

LC/MS (m/z) 364.1 (M⁺); RT=3.87, UV purity=95.4, ELS purity=99.2.

### {4-[(3-Chlorophenylamino)methyl]-2-nitrophenyl}carbamic acid ethyl ester

LC/MS (m/z) 351.2 (M⁺); RT=3.58, UV purity=92.3, ELS purity=98.7. ¹H NMR (CDCl₃): 1.35 (t, 3H); 4.18-4.30 (m, 3H); 4.38 (s, 2H); 6.45 (d, 1H); 6.55 (s, 1); 6.70 (d, 1H); 7.05 (m, 1H); 7.65 (d, 1H); 8.20 (s, 1H); 8.55 (d, 1H); 9.80 (br s, 1H).

### [2-Nitro-4-(m-tolylaminomethyl)phenyl]carbamic acid ethyl ester

LC/MS (m/z) 330.1 (M⁺); RT=3.20, UV purity=96.6, ELS purity=98.2. ¹H NMR (CDCl₃): 1.35 (t, 3H); 2.25 (s, 3H); 4.10 (m, 1H); 4.25 (q, 2H); 4.38 (m, 2H); 6.40 (d, 1H); 6.45 (s, 1H); 6.60 (d, 1H); 7.05 (m, 1H); 7.65 (d, 1H); 8.20 (s, 1H); 8.55 (d, 1H); 9.80 (br s, 1H).

### {4-[1-(4-Chlorophenylamino)ethyl]-2-nitrophenyl}carbamic acid ethyl ester

LC/MS (m/z) 363.2 (M⁺); RT=3.68, UV purity=93.6, ELS purity=98.0. ¹H NMR (CDCl₃): 1.35 (t, 3H); 1.55 (d, 3H); 4.10 (s, 1H); 4.22 (q, 2H); 4.45 (q, 1H); 6.38 (d, 2H); 7.05 (d, 2H); 7.60 (d, 1H); 8.15 (s, 1H); 8.55 (d, 1H); 9.72 (s, 1H).

### {2-Nitro-4-[1-(4-trifluoromethylphenylamino)ethyl]phenyl}carbamic acid ethyl ester

LC/MS (m/z) 397.6 (M⁺); RT=3.73, WV purity=97.7, ELS purity=99.8. ¹H NMR (CDCl₃): 1.35 (t, 3H); 1.50 (d, 3H); 4.10 (q, 2H); 4.35 (d, 1H); 4.50 (m, 1H); 6.15 (d, 2H); 7.00 (d, 2H); 7.50 (d, 1H); 8.15 (s, 1H); 8.55 (d, 1H); 9.72 (s, 1H).

### N-{4-[(3-Fluorophenylamino)methyl]-2-nitrophenyl}-2,2-dimethylpropionamide

¹H NMR (CDCl₃): 1.35 (s, 9H); 4.30 (br m, 1H); 4.40 (d, 2H); 6.25 (d, 1H); 6.35 (d, 1H); 6.42 (br m, 1H); 7.08 (m, 1H); 7.65 (d, 1H); 8.20 (s, 1H); 8.80 (d, 1H); 10.70 (br s, 1H).

Compounds of the Invention

### Example 1

### 1a {2-Amino-4-[(4-tert-butylphenylamino)-methyl]-phenyl}-carbamic acid ethyl ester

{4-[(4-tert-Butylphenylamino)-methyl]-2-nitrophenyl}-carbamic acid ethyl ester (400 mg, 1.08 mmol) was dissolved in tetrahydrofuran (20 mL) and heated to 40° C. Sodiumdithionite (1.13 g, 6.5 mmol) dissolved in water (20 mL) was added. The mixture was stirred vigorously at 40° C. until all starting material was consumed as judged by TLC. After cooling to ambient temperature brine (10 mL) is added and the mixture extracted with tetrahydrofuran (2×15 mL). The combined organic phases are dried over MgSO₄, filtered and evaporated to dryness in vacuo. Purified by chromatography using silica gel on a Flashmaster system eluting with heptane/ethyl acetate (linear gradient from 1:0 to 4:1). Fractions containing the product were pooled and evaporated in vacuo to yield the title compound as an off-white solid (230 mg, 62%). LC/MS (m/z) 341.1 (M⁺); RT=2.12, UV purity=97.8, ELS purity=99.1. ¹H NMR (CDCl₃): 1.27 (s, 9H); 1.32 (t, 3H); 3.75 (br, 2H, NH₂; 3.80 (br s, 1H, NH); 4.22 (br m, 4H); 6.22 (br s, 1H, NH); 6.55 (d, 2H); 6.80 (m, 2H); 7.20 (br m, 3H).

The following compounds were prepared analogously:

### 1b (2-Amino-4-phenylaminoethyl-phenyl)-carbamic acid ethyl ester

LC/MS (m/z) 285.1 (M⁺); RT=1.46, UV purity=98.2, ELS purity=99.5. ¹H NMR (CDCl₃): 1.30 (t, 3H); 3.75 (br s, 2H, NH₂); 3.95 (br s, 1H, NH); 4.22 (br m, 4H); 6.25 (br s, 1H, NH); 6.60 (d, 2H); 6.72 (t, 1H); 6.82 (m, 2H); 7.15 (t, 2H); 7.22 (br m, 1H).

### 1c [2-Amino-4-(naphthalen-2-ylaminomethyl)-phenyl]-carbamic acid ethyl ester

LC/MS (m/z) 336.2 (MH⁺); RT=2.20, UV purity=98.2, ELS purity=99.4. ¹H NMR (CDCl₃): 1.31 (t, 3H); 3.80 (br, 3H, NH₂+NH); 4.25 (q, 2H); 4.35 (s, 2H); 6.25 (br s, 1H, NH); 6.82 (m, 3H); 6.92 (m, 1H); 7.18 (m, 1H); 7.22 (br m, 1H); 7.35 (m, 1H); 7.65 (br m, 3H).

### 1d [2-Amino-4-(p-tolylamino-methyl)-phenyl]-carbamic acid ethyl ester

LC/MS (m/z) 298.1 (M⁺); RT=1.50, WV purity=98.3, ELS purity=98.4. ¹H NMR (CDCl₃): 1.30 (t, 3H); 2.22 (s, 3H); 3.78 (br, 3H, NH₂+NH); 4.22 (br m, 4H); 6.25 (br s, 1H, NH); 6.55 (d, 2H); 6.80 (m, 2H); 6.98 (d, 2H); 7.21 (br m, 1H).

### 1e {2-Amino-4-[(4-trifluoromethylphenylamino)-methyl]-phenyl}-carbamic acid ethyl ester

LC/MS (m/z) 353.1 (M⁺); RT=2.58, UV purity=97.9, ELS purity=99.2. ¹H NMR (CDCl₃): 1.30 (t, 3H); 3.76 (br s, 2H, NH₂); 4.23 (br m, 4H); 4.40 (br s, 1H, NH); 6.28 (br s, 1H, NH); 6.60 (d, 2H); 6.75 (m, 2H); 7.20 (br m, 1H); 7.40 (d, 2H).

### 1f {2-Amino-4-[(4-chlorophenylamino)-methyl]-phenyl}-carbamic acid ethyl ester

LC/MS (m/z) 319.0 (MH⁺); RT=2.24, UV purity=98.9, ELS purity=98.7. ¹H NMR (CDCl₃): 1.31 (t, 3H); 3.76 (br s, 2H, NH₂); 4.00 (br s, 1H, NH); 4.22 (br m, 4H); 6.23 (br s, 1H, NH); 6.52 (d, 2H); 6.76 (m, 2H); 7.10 (d, 2H); 7.22 (br m, 1H).

### 1g {2-Amino-4-[(3-fluorophenylamino)-methyl]-phenyl}-carbamic acid ethyl ester

LC/MS (m/z) 303.1 (M⁺); RT=2.08, UV purity=98.5, ELS purity=99.9. ¹H NMR (CDCl₃): 1.32 (t, 3H); 3.75 (br s, 2H, NH₂); 4.15 (br s, 1H, NH); 4.24 (br m, 4H); 6.20 (br s, 1H, NH); 6.30 (m, 1H); 6.38 (m, 2H); 6.78 (m, 2H); 7.08 (m, 1H); 7.22 (br m, 1H).

### 1h {2-Amino-4-[(4-fluorophenylamino)-methyl]-phenyl}-carbamic acid ethyl ester

LC/MS (m/z) 304.2 (M⁺); RT=1.58, UV purity=96.1, ELS purity=98.8.11H NMR (CDCl₃): 1.32 (t, 3H); 3.82 (br s, 3H, NH+NH₂); 4.18 (s, 2H); 4.23 (q, 2H); 6.25 (br s, 1H, NH); 6.52 (m, 2H); 6.77 (m, 2H); 6.88 (m, 2H); 7.20 (br m, 1H).

### 1i {2-Amino-4-[(2-fluorophenylamino)-methyl]-phenyl}-carbamic acid ethyl ester

LC/MS (m/z) 303.0 (M⁺); RT=2.16, UV purity=99.5, ELS purity=99.8. ¹H NMR (CDCl₃): 1.30 (t, 3H); 3.75 (br s, 2×, NH₂); 4.21 (q, 2H); 4.28 (s, 2H); 4.38 (br s, 1H, NH); 6.30 (br s, 1H, NH); 6.63 (m, 2H); 6.70 (m, 2H); 6.95 (m, 2H); 7.20 (br m, 1H).

### 1j [2-Amino-4-(biphenyl-4-ylaminomethyl)-phenyl]-carbamic acid ethyl ester

LC/MS (m/z) 361.2 (M⁺); RT=2.45, UV purity=97.0, ELS purity=98.3. ¹H NMR (CDCl₃): 1.32 (t, 3H); 3.90 (br s, 3H, NH+NH₂); 4.21 (q, 2H); 4.30 (s, 2H); 6.25 (br s, 1H, NH); 6.70 (m, 2H); 6.82 (m, 2H); 7.25 (m, 2H); 7.37 (m, 2H); 7.44 m, 2H); 7.55 (m, 2H).

### 1k {2-Amino-4-[(2,4-difluorophenylamino)-methyl]-phenyl}-carbamic acid ethyl ester

LC/MS (m/z) 320.1 (M⁺); RT=2.24, UV purity=95.9, ELS purity=99.9. ¹H NMR (CDCl₃): 1.31 (t, 3H); 3.75 (br s, 2H, NH₂); 4.12 (br s, 1H, NH); 4.23 (br m, 4H); 6.27 (br s, 1H, NH); 6.55 (m, 1H); 6.70 (m, 1H); 6.78 (m, 3H); 7.22 (br m, 1H).

### 1l {2-Amino-4-[(4-methoxyphenylamino)-methyl]-phenyl}-carbamic acid ethyl ester

LC/MS (m/z) 314.9 (M⁺); RT=1.29, UV purity=95.6, ELS purity=99.9. ¹H NMR (CDCl₃): 1.31 (t, 3H); 3.72 (br m, 6H, OCH₃+NH+NH₂); 4.18 (s, 2H); 4.24 (q, 2H); 6.30 (br s, 1H, NH); 6.60 (d, 2H); 6.78 (br m, 4H); 7.21 (br m, 1H).

### 1m {2-Amino-4-[(4-cyclohexylphenylamino)-methyl]-phenyl}-carbamic acid ethyl ester

LC/MS (m/z) 366.9 (MH⁺); RT=2.45, WV purity=96.2, ELS purity=99.5. ¹H NMR (CDCl₃): 1.30 (br m, 9H); 1.82 (m, 4H); 2.40 (m, 1H); 3.78 (br, 3H, NH₂+NH); 4.22 (br m, 4H); 6.25 (br s, 1H, NH); 6.57 (d, 1H); 6.62 (d, 1H); 6.80 (m, 2H); 7.02 (m, 2H); 7.20 (br m, 1H).

### 1n [2-Amino-4-(indan-5-ylaminomethyl)-phenyl]-carbamic acid ethyl ester

LC/MS (m/z) 325.2 (M⁺); RT=1.75, WV purity=96.1, ELS purity=98.4. ¹H NMR (CDCl₃): 1.30 (t, 3H); 2.02 (m, 2H); 2.80 (m, 4H); 3.75 (br, 3H, NH₂+NH); 4.22 (br m, 4H); 6.27 (br s, 1H, NH); 6.42 (d, 1H); 6.55 (s, 1H); 6.80 (m, 2H); 7.00 (d, 1H); 7.21 (br m, 1H).

### 1o {2-Amino-4-[(4-isopropylphenylamino)-methyl]-phenyl}-carbamic acid ethyl ester

LC/MS (m/z) 326.2 (MH⁺); RT=1.91, WV purity=95.2, ELS purity=98.5. ¹H NMR (CDCl₃): 1.20 (d, 6H), 1.32 (t, 3H); 2.80 (m, 1H); 3.75 (br, 3H, NH₂+NH); 4.22 (br m, 4H); 6.25 (br s, 1H, NH); 6.57 (d, 2H); 6.81 (m, 2H); 7.05 (d, 2H); 7.20 (br m, 1H).

### 1p {2-Amino-4-[(4-butylphenylamino)-methyl]-phenyl}-carbamic acid ethyl ester

LC/MS (m/z) 340.2 (M⁺); RT=2.16, UV purity=97.2, ELS purity=99.5. ¹H NMR (CDCl₃): 0.90 (t, 3H); 1.32 (m, 5H); 1.55 (m, 2H); 2.52 (t, 2H); 3.72 (br, 2H, NH₂); 3.88 (br, 1H, NH); 4.21 (br m, 4H); 6.22 (br s, 1H, NH); 6.56 (d, 2H); 6.80 (m, 2H); 6.98 (d, 2H); 7.20 (br m, 1H).

### Example 2

### 1q {2-Amino-4-[(4-chloro-3-fluorophenylamino)methyl]phenyl}carbamic acid ethyl ester

A suspension of {4-[2-(4-Chloro-3-fluorophenyl)ethyl]-2-nitrophenyl}carbamic acid ethyl ester (1.80 g, 4.89 mmol), zinc (1.96 g, 29.4 mmol) and ammonium chloride (2.60, 48.9 mmol) was refluxed in methanol (50 mL) for 3 hours. The mixture was filtered off and purified by chromatography using silica gel on a Flashmaster system eluting with heptane/ethyl acetate (linear gradient from 1:0 to 5:1). Fractions containing the product were pooled and evaporation in vacuo gave the title compound as an off-white solid (1.27 g, 77%).

LC/MS (m/z) 337.3 (M⁺); RT=2.58, UV purity=95.7, ELS purity=98.3. ¹H NMR (CDCl₃): 1.30 (t, 3H); 3.80 (br, 2H); 4.20 (q, 2H); 4.35 (br m, 3H); 6.35 (br m, 3H); 6.75 (m, 2H); 7.10 (m, 1H); 7.22 (m, 1H).

The following compounds were prepared analogously:

### 1r {2-Amino-4-[(2,4-dichlorophenylamino)methyl]phenyl}carbamic acid ethyl ester

LC/MS (m/z) 355.1 (M⁺); RT=2.83, UV purity=99.5, ELS purity=99.7. ¹H NMR (CDCl₃): 1.31 (t, 3H); 3.77 (br s, 2H); 4.20 (q, 2H); 4.28 (br m, 2H); 4.68 (br t, 1H); 6.27 (br s, 1H); 6.50 (d, 1H); 6.75 (m, 2H); 7.05 (d, 1H); 7.22 (br m, 2H).

### 1s {2-Amino-4-[(2,3-dichlorophenylamino)methyl]phenyl}carbamic acid ethyl ester

LC/MS (m/z) 355.0 (M⁺); RT=2.79, UV purity=95.9, ELS purity=99.8. ¹H NMR (CDCl₃): 1.30 (t, 3H); 3.75 (br s, 2H); 4.20 (q, 2H); 4.30 (br m, 2H); 4.85 (br m, 1H); 6.30 (br s, 1H); 6.48 (d, 1H); 6.72-6.80 (m, 3H); 7.05 (m, 1H); 7.22 (br d, 1H).

### 1t {2-Amino-4-[(3,5-dichlorophenylamino)methyl]phenyl}carbamic acid ethyl ester

LC/MS (m/z) 354.9 (M⁺); RT=2.85, UV purity=98.5, ELS purity=99.4. ¹H NMR (CDCl₃): 1.30 (t, 3H); 3.78 (br s, 2H); 4.15 (m, 2H); 4.25 (br m, 3H); 6.30 (br s, 1H); 6.45 (s, 2H); 6.65 (s, 1H); 6.75 (m, 2H); 7.22 (br d, 1H).

### 1u {2-Amino-4-[(3,4-dichlorophenylamino)methyl]phenyl}carbamic acid ethyl ester

LC/MS (m/z) 354.0 (M⁺); RT=2.66, UV purity=94.5, ELS purity=99.9. ¹H NMR (CDCl₃): 1.30 (t, 3H); 3.75 (br s, 2H); 4.15 (br m, 3H); 4.22 (q, 2H); 6.27 (br s, 1H); 6.45 (d, 1H); 6.65 (s, 1H); 6.75 (m, 2H); 7.12 (d, 1H); 7.20 (br d, 1H).

### 1v {2-Amino-4-[(3-trifluoromethylphenylamino)methyl]phenyl}carbamic acid ethyl ester

LC/MS (m/z) 353.1 (M⁺); RT=2.66, UV purity=98.8, ELS purity=99.9. ¹H NMR (CDCl₃): 1.30 (t, 3H); 3.75 (br s, 2H); 4.18-4.28 (br m, 5H); 6.32 (br s, 1H); 6.70-6.80 (br m, 3H); 6.82 (s, 1H); 6.90 (d, 1H); 7.20 (br m, 2H).

### 1x {2-Amino-4-[(3-fluoro-4-trifluoromethylphenylamino)methyl]phenyl}carbamic acid ethyl ester

LC/MS (m/z) 371.1 (M⁺); RT=2.74, UV purity=95.2, ELS purity=99.5. ¹H NMR (CDCl₃): 1.30 (t, 3H); 3.70 (br, 2H); 4.10 (m, 4H); 4.50 (br, 1H); 6.30 (br m, 3H); 6.60 (m, 2H); 7.20 (m, 2H).

### 1y {2-Amino-4-[(3,4-difluorophenylamino)methyl]phenyl}carbamic acid ethyl ester

LC/MS (m/z) 321.1 (M⁺); RT=2.24, UV purity=95.3, ELS purity=98.0. ¹H NMR (CDCl₃): 1.30 (t, 3H); 3.80 (br, 3H); 4.15 (s, 2H); 4.25 (q, 2H); 6.22 (m, 1H); 6.40 (br s, 1H); 6.45 (m, 1H); 6.72 (m, 2H); 6.90 (m, 1H); 7.20 (d, 1H).

### 1z {2-Amino-4-[(4-cyanophenylamino)methyl]phenyl}carbamic acid ethyl ester

LC/MS (m/z) 310.0 (M⁺); RT=2.04, UV purity=97.8, ELS purity=99.3. ¹H NMR (CDCl₃): 1.30 (t, 3H); 3.75 (br, 2H); 4.20 (m, 4H); 4.65 (br, 1H); 6.35 (br, 1H); 6.55 (d, 2H); 6.72 (d, 2H); 7.20 (m, 1H); 7.50 (m, 2H).

### 1aa {2-Amino-4-[(4-fluoro-3-trifluoromethylphenylamino)methyl]phenyl}carbamic acid ethyl ester

LC/MS (m/z) 371.2 (M⁺); RT=2.70, UV purity=98.0, ELS purity=98.8. ¹H NMR (CDCl₃): 1.30 (t, 3H); 3.90 (br, 3H); 4.15 (s, 2H); 4.25 (q, 2H); 6.40 (br s, 1H); 6.60 (m, 1H); 6.75 (m, 3H); 6.95 (m, 1H); 7.20 (d, 1H).

### 1ba {2-Amino-4-[(3-chloro-4-methylphenylamino)methyl]phenyl}carbamic acid ethyl ester

LC/MS (m/z) 334.0 (M⁺); RT=2.49, UV purity=97.0, ELS purity=99.6. ¹H NMR (CDCl₃): 1.30 (t, 3H); 2.20 (s, 3H); 3.75 (br, 3H); 4.15 (s, 2H); 4.25 (q, 2H); 6.30 (br, 1H); 6.40 (d, 1H); 6.65 (s, 1H); 6.72 (d, 2H); 6.98 (d, 1H); 7.20 (d, 1H).

### 1ca {2-Amino-4-[(3-chlorophenylamino)methyl]phenyl}carbamic acid ethyl ester

LC/MS (m/z) 320.0 (M⁺); RT=2.33, UV purity=97.5, ELS purity=99.7. ¹H NMR (CDCl₃): 1.30 (t, 3H); 3.75 (br, 3H); 4.15 (s, 2H); 4.20 (q, 2H); 6.40-6.50 (br, 2H); 6.55 (s, 1H); 6.65-6.80 (br, 3H); 7.00 (m, 1H); 7.18 (d, 1H).

### 1da [2-Amino-4-(m-tolylaminomethyl)phenyl]carbamic acid ethyl ester

LC/MS (m/z) 298.1 (M⁺); RT=1.66, UV purity=95.0, ELS purity=99.9. ¹H NMR (CDCl₃): 1.30 (t, 3H); 2.22 (s, 3H); 3.75 (br, 3H); 4.20 (m, 4H); 6.40 (br, 3H); 6.50 (d, 1H); 6.72 (br, 2H); 7.00 (m, 1H); 7.15 (d, 1H).

### 1ea {2-Amino-4-[1-(4-chlorophenylamino)ethyl]phenyl}carbamic acid ethyl ester

LC/MS (m/z) 333.5 (M⁺); RT=2.37, UV purity=98.1, ELS purity=99.4. ¹H NMR (CDCl₃): 1.30 (t, 3H); 1.45 (d, 3H); 3.70 (br, 2H); 3.95 (br, 1H); 4.20 (q, 2H); 4.40 (q, 1H); 6.25 (br, 1H); 6.40 (d, 2H); 6.75 (m, 2H); 7.00 (d, 2H); 7.20 (d, 1H).

### 1fa {2-Amino-4-[1-(4-trifluoromethylphenylamino)ethyl]phenyl}carbamic acid ethyl ester

LC/MS (m/z) 368.2 (M⁺); RT=2.70, UV purity=99.8, ELS purity=97.7. ¹H NMR (CDCl₃): 1.20 (t, 3H); 1.40 (d, 3H); 4.05 (q, 2H); 4.30 (q, 1H); 4.80 (br, 2H); 6.55 (br, 3H); 6.63 (s, 1H); 6.80 (d, 1H); 7.10 (d, 1H); 7.30 (d, 2H); 8.45 (br, 1H).

### 1cb N-{2-Amino-4-[(3-fluorophenylamino)methyl]phenyl}-2,2-dimethylpropionamide

LC/MS (m/z) 316.5 (M⁺); RT=2.15, UV purity=94.6, ELS purity=99.7. ¹H NMR (CDCl₃): 1.35 (s, 9H); 3.80 (br s, 2H); 4.15 (br s, 1H); 4.25 (br m, 2H); 6.28 (d, 1H); 6.40 (m, 2H); 6.78 (m, 2H); 7.08 (m, 1H); 7.15 (d, 1H); 7.30 (br s, 1H).

### Example 3

### 1ga {4-[(4-Chlorophenylamino)methyl]phenyl}carbamic acid ethyl ester

A solution of (4-formylphenyl)carbamic acid ethyl ester (0.50 g, 2.59 mmol) and 4-chloroaniline (0.43 g, 3.36 mmol) in dry ethanol (10 mL) was refluxed for 12 hours, cooled on an ice bath and the precipitated imine filtered off. It was washed once with cold water and suspended in methanol (10 mL) and acetic acid (1 mL). Sodium cyanoborohydride (0.42 g, 6.75 mmol) was added and the mixture stirred 1 hour at ambient temperature. A second portion of sodium cyanoborohydride (0.42 g, 6.75 mmol) was added and the mixture stirred 3 hours at ambient temperature. A saturated solution of sodium hydrogencarbonate (50 mL) was added and the precipitate filtered off and washed twice with water. Purified by chromatography using silica gel on a Flashmaster system eluting with heptane/ethyl acetate (linear gradient from 1:0 to 5:1). Fractions containing the product were pooled and evaporation in vacuo gave the title compound as a white solid (0.50 g, 73%).

LC/MS (m/z) 305.8 (M⁺); RT=2.95, UV purity=97.2, ELS purity=99.5. ¹H NMR (CDCl₃): 1.35 (t, 3H), 4.05 (br s, 1H); 4.25 (br m, 4H); 6.60 (br m, 3H); 7.15 (d, 2H); 7.35 (br m, 4H). ¹³C NMR (CDCl₃): 15.0, 48.3, 61.7, 114.3, 119.3, 122.5, 128.6, 129.5, 134.2, 137.6, 147.0, 154.0.

### 1ha {4-[(4-Trifluoromethylphenylamino)methyl]phenyl}carbamic acid ethyl ester

LC/MS (m/z) 339.4 (M⁺); RT=3.45, UV purity=97.2, ELS purity=98.3. ¹H NMR (CDCl₃): 1.35 (t, 3H); 4.25 (q, 2H); 4.35 (br m, 3H); 6.55 (br s, 1H); 6.65 (d, 2H); 7.30-7.45 (br m, 6H). ¹³C NMR (CDCl₃): 14.9, 47.7, 61.7, 112.4, 119.4, 122.5, 127.0, 128.5, 134.2, 137.6, 150.8, 154.0.

### 1ia {4-[1-(4-Chlorophenylamino)ethyl]phenyl}carbamic acid ethyl ester

LC/MS (m/z) 318.1 (M⁺); RT=3.03, UV purity=97.5, ELS purity=98.1. ¹H NMR (CDCl₃): 1.28 (t, 3H); 1.47 (d, 3H); 4.00 (br, 1H); 4.20 (q, 2H); 4.40 (q, 1H); 6.40 (d, 2H); 6.55 (br, 1H); 7.00 (d, 2H); 7.20 (d, 2H); 7.33 (d, 2H).

### 1ja {4-[(4-Fluorophenylamino)methyl]-2-methylphenyl}carbamic acid ethyl ester

LC/MS (m/z) 302.1 (M⁺); RT=2.20, UV purity=95.0, ELS purity=97.3. ¹H NMR (CDCl₃): 1.30 (t, 3H); 2.25 (s, 3H); 4.0 (br s, 1H); 4.25 (br m, 4H); 6.35 (br s, 1H); 6.55 (br m, 2H); 6.87 (br m, 2H); 7.17 (br m, 2H); 7.75 (br d, 1H). ¹³C NMR (CDCl₃): 14.6, 17.7, 48.6, 61.3, 113.8, 115.6, 126.1, 129.6, 135.1, 144.3, 156.9.

### 1ka {4-[(4-Chlorophenylamino)methyl]-2-methylphenyl}carbamic acid ethyl ester

LC/MS (m/z) 318.1 (M⁺); RT=3.12, UV purity=98.6, ELS purity=99.8. ¹H NMR (CDCl₃): 1.30 (t, 3H); 2.25 (s, 3H); 4.10 (br s, 1H); 4.25 (br m, 4H); 6.35 (br s, 1H); 6.55 (d, 2H); 7.15 (br m, 4H); 7.75 (br d, 1H). ¹³C NMR (CDCl₃): 14.6, 17.7, 48.1, 61.3, 114.1, 122.3, 126.0, 129.1, 129.5, 134.3, 135.2, 146.4, 153.9.

### 1la {2-Methyl-4[(4-trifluoromethylphenylamino)methyl]phenyl}carbamic acid ethyl ester

LC/MS (m/z) 352.5 (M⁺); RT=3.45, UV purity=96.7, ELS purity=99.9. ¹H ¹NMR (CDCl₃): 1.30 (t, 3H); 2.25 (s, 3H); 4.25 (br m, 4H); 4.40 (br s, 1H); 6.35 (br s, 1H); 6.62 (d, 2H); 7.15 (br m, 2H); 7.45 (d, 2H); 7.75 (br d, 1H). ¹³C NMR (CDCl₃): 14.6, 17.7, 47.5, 61.4, 112.1, 123.9, 126.0, 126.6, 129.5, 135.3, 150.3, 153.9.

### 1ma {4-[(3,4-Difluorophenylamino)methyl]-2-methylphenyl}carbamic acid ethyl ester

LC/MS (m/z) 320.2 (M⁺); RT=3.12, UV purity=95.6, ELS purity=99.1. ¹H NMR (CDCl₃): 1.30 (t, 3H); 2.25 (s, 3H); 4.00 (br s, 1H); 4.20 (br m, 4H); 6.20-6.35 (br m, 3H); 6.95 (m, 1H); 7.15 (br m, 2H); 7.75 (br d, 1H). ¹³C NMR (CDCl₃): 14.6, 17.7, 48.3, 61.4, 101.5, 108.1, 117.4, 121.4, 126.0, 129.5, 129.9, 134.1, 135.3, 142.1, 144.9, 149.9, 153.9.

### 1na {4-[(3-Fluorophenylamino)methyl]-2-methylphenyl}carbamic acid ethyl ester

LC/MS (m/z) 302.4 (M⁺); RT=3.08, UV purity=96.7, ELS purity=98.0.11H NMR (CDCl₃): 1.30 (t, 3H); 2.25 (s, 3H); 4.25 (br m, 5H); 6.35 (br m, 4H); 7.00-7.20 (br m, 3H); 7.75 (br d, 1H). ¹³C NMR (CDCl₃): 14.6, 17.7, 47.8, 61.3, 99.6, 104.1, 108.8, 121.4, 126.1, 129.6, 130.2, 130.3, 134.3, 135.2, 149.7, 153.9, 163.1, 165.0.

### 1oa {2-Chloro-4-[(4-chlorophenylamino)methyl]phenyl}carbamic acid ethyl ester

LC/MS (m/z) 339.1 (M⁺); RT=3.58, UV purity=98.3, ELS purity=99.9. ¹H NMR (CDCl₃): 1.35 (t, 3H); 4.10 (br, 1H); 4.30 (br, 4H); 6.55 (d, 2H); 7.10 (br m, 3H); 7.22 (d, 1H); 7.40 (s, 1H); 8.15 (d, 1H).

### 1pa {2-Chloro-4-[(4-trifluoromethylphenylamino)methyl]phenyl}carbamic acid ethyl ester

LC/MS (m/z) 372.3 (M⁺); RT=3.74, UV purity=96.5, ELS purity=99.8. ¹H NMR (CDCl₃): 1.35 (t, 3H); 4.25 (q, 2H); 4.35 (s, 2H); 4.45 (br, 1H); 6.65 (d, 2H); 7.10 (br, 1H); 7.22 (d, 1H); 7.35 (s, 1H); 7.45 (d, 2H); 8.20 (d, 1H).

### 1qa {2-Chloro-4-[(4-fluorophenylamino)methyl]phenyl}carbamic acid ethyl ester

LC/MS (m/z) 323.1 (M⁺); RT=2.91, UV purity=99.7, ELS purity=96.7. ¹H NMR (CDCl₃): 1.32 (t, 3H); 4.10 (br s, 1H); 4.25 (br m, 4H); 6.55 (m, 2H); 6.88 (m, 2H); 7.10 (br s, 1H); 7.22 (d, 1H); 7.47 (s, 1H); 8.15 (d, 1H).

### 1ra {2-Chloro-4-[(3-fluorophenylamino)methyl]phenyl}carbamic acid ethyl ester

LC/MS (m/z) 322.6 (M⁺); RT=3.37, UV purity=99.9, ELS purity=99.9. ¹H NMR (CDCl₃): 1.33 (t, 3H); 4.00 (br s, 1H); 4.25 (br m, 4H); 6.28 (d, 1H); 6.40 (m, 2H); 7.08 (m, 2H); 7.22 (d, 1H); 7.37 (s, 1H); 8.15 (d, 1H).

### 1sa {2-Chloro-4-[(3,4-dichlorophenylamino)methyl]phenyl}carbamic acid ethyl ester

LC/MS (m/z) 374.0 (M⁺); RT=3.78, UV purity=99.9, ELS purity=97.6. ¹H NMR (CDCl₃): 1.35 (t, 3H); 4.15 (br s, 1H); 4.25 (br m, 4H); 6.45 (d, 1H); 6.70 (s, 1H); 7.10 (br s, 1H); 7.15 (d, 1H); 7.20 (d, 1H); 7.35 (s, 1H); 8.15 (d, 1H).

### 1ta {2-Chloro-4-[(4-chloro-3-fluorophenylamino)methyl]phenyl}carbamic acid ethyl ester

LC/MS (m/z) 358.0 (M⁺); RT=3.62, UV purity=99.9, ELS purity=96.4. ¹H NMR (CDCl₃): 1.32 (t, 3H); 4.15 (br s, 1H); 4.25 (br m, 4H); 6.35 (m, 2H); 7.10 (m, 2H); 7.22 (d, 1H); 7.35 (s, 1H); 8.15 (d, 1H).

### 1ua {4-[(4-Chlorophenylamino)methyl]-2-fluorophenyl}carbamic acid ethyl ester

LC/MS (m/z) 323.1 (M⁺); RT=3.24, UV purity=99.9, ELS purity=99.9. ¹H NMR (CDCl₃): 1.32 (t, 3H); 4.15 (br s, 1H); 4.23 (br m, 4H); 6.50 (m, 2H); 6.78 (br s, 1H); 7.10 (m, 4H); 8.05 (br m, 1H).

### 1va {4-[(4-chloro-3-fluorophenylamino)methyl]-2-fluorophenyl}carbamic acid ethyl ester

LC/MS (m/z) 340.6 (M⁺); RT=3.37, UV purity=96.8, ELS purity=99.9. ¹H NMR (CDCl₃): 1.32 (t, 3H); 4.10 (br s, 1H); 4.25 (br m, 4H); 6.35 (m, 2H); 6.75 (br s, 1H); 7.10 (m, 3H); 8.07 (br m, 1H).

### 1xa {2-Fluoro-4-[(4-trifluoromethylphenylamino)methyl]phenyl}carbamic acid ethyl ester

LC/MS (m/z) 356.2 (M⁺); RT=3.45, WV purity=97.1, ELS purity=97.8. ¹H NMR (CDCl₃): 1.32 (t, 3H); 4.22 (q, 2H); 4.32 (s, 2); 4.40 (s, 1H); 6.60 (d, 2H); 6.78 (br s, 1H); 7.08 (m, 2H); 7.40 (d, 2H); 8.05 (br m, 1H).

### 1ya {4′-Dimethylamino-5-[(3-fluorophenylamino)methyl]biphenyl-2-yl}-4-carbamic acid ethyl ester

LC/MS (m/z) 408.0 (M⁺); RT=2.49, UV purity=97.9, ELS purity=99.7. ¹H NMR (CDCl₃): 1.25 (t, 3H); 3.00 (s, 6H); 4.10 (br m, 1H); 4.17 (q, 2H); 4.28 (br s, 2H); 6.30 (d, 1H); 6.38 (m, 2H); 6.75 (br s, 1H); 6.83 (d, 2H); 7.08 (m, 1H); 7.17 (s, 1H); 7.22 (m, 2H); 7.30 (d, 1H); 8.10 (br m, 1H).

### 1za {4′-Dimethylamino-5-[(4-trifluoromethylphenylamino)methyl]biphenyl-2-yl}-carbamic acid ethyl ester

LC/MS (m/z) 458.0 (M⁺); RT=3.09, UV purity=97.2, ELS purity=99.7. ¹H NMR (CDCl₃): 1.25 (t, 3H); 3.00 (s, 6H); 4.10 (br m, 1H); 4.17 (q, 2H); 4.28 (br s, 2H); 6.38 (m, 2H); 6.62 (d, 2H); 6.75 (br s, 1H); 6.83 (d, 2H); 7.17 (s, 1H); 7.30 (d, 1H); 7.40 (d, 2H); 8.10 (br m, 1H).

### 1ab {4′-Chloro-5-[(3-fluorophenylamino)methyl]biphenyl-2-yl}carbamic acid ethyl ester

LC/MS (m/z) 397.4 (M⁺); RT=4.22, UV purity=97.3, ELS purity=98.7. ¹H NMR (CDCl₃): 1.25 (t, 3H); 3.90 (br m, 1H); 4.15 (q, 2H); 4.35 (br s, 2H); 6.30 (d, 1H); 6.36 (m, 2H); 6.50 (br s, 1H); 7.05 (m, 1H); 7.20 (s, 1H); 7.22 (s, 1H); 7.30 (d, 1H); 7.40 (d, 1H); 7.50 (d, 2H); 8.10 (br m, 1H).

### 1bb {4′-Chloro-5-[(4-trifluoromethylphenylamino)methyl]biphenyl-2-yl}carbamic acid ethyl ester

LC/MS (m/z) 449.2 (M⁺); RT=4.09, UV purity=97.7, ELS purity=91.9. ¹H NMR (CDCl₃): 1.27 (t, 3H); 3.95 (br m, 1H); 4.15 (q, 2H); 4.35 (br m, 2H); 6.47 (br s, 1H); 6.62 (d, 2H); 7.15 (s, 1H); 7.28 (d, 2H); 7.35 (d, 1H); 7.40 (d, 2H); 7.48 (d, 2H); 8.10 (br m, 1H).

### 1db N-{4-[(4-Chlorophenylamino)methyl]phenyl}butyramide

LC/MS (m/z) 303.3 (M⁺); RT=2.73, UV purity=96.6, ELS purity=99.9. ¹H NMR (CDCl₃): 1.00 (t, 3H); 1.75 (m, 2H); 2.32 (t, 2H); 4.19 (br s, 1H); 4.25 (s, 2H); 6.55 (d, 2H); 7.05 (br s, sH); 7.10 (d, 2H); 7.30 (d, 2H); 7.50 (d, 2H).

### 1eb N-{4-[(3,4-Dichlorophenylamino)methyl]phenyl}butyramide

LC/MS (m/z) 337.6 (M⁺); RT=3.22, UV purity=96.0, ELS purity=99.6. ¹H NMR (CDCl₃): 1.00 (t, 3H); 1.77 (m, 2H); 2.32 (t, 2H); 4.10 (br s, 1H); 4.25 (s, 2H); 6.45 (d, 1H); 6.70 (s, 1H); 7.10 (br s, 1H); 7.15 (d, 1H); 7.30 (d, 2H); 7.50 (d, 2H).

### 1fb N-{4-[(4-Chloro-3-fluorophenylamino)methyl]phenyl}butyramide

LC/MS (m/z) 320.9 (M⁺); RT=3.08, UV purity=96.9, ELS purity=99.6. ¹H NMR (CDCl₃): 1.03 (t, 3H); 1.75 (m, 2H); 2.32 (t, 2H); 4.15 (br s, 1H); 4.22 (s, 2H); 6.32 (m, 1H); 6.40 (m, 1H); 7.10 (m, 2H); 7.30 (d, 2H); 7.55 (d, 2H).

### 1gb N-{4[(4-fluorophenylamino)methyl]-2-methylphenyl}butyramide

LC/MS (m/z) 300.6 (M⁺); RT=1.89, UV purity=99.5, ELS purity=99.9. ¹H NMR (CDCl₃): 1.05 (t, 3H); 1.78 (m, 2H); 2.22 (s, 3H); 2.38 (t, 2H); 4.00 (br, 1H); 4.20 (s, 2H); 6.55 (m, 2H); 6.90 (br m, 3H); 7.18 (m, 2H); 7.80 (d, 1H).

### 1hb N-{4[(3-fluorophenylamino)methyl]-2-methylphenyl}butyramide

LC/MS (m/z) 300.5 (M⁺); RT=2.79, WV purity=99.5, ELS purity=99.9. ¹H NMR (CDCl₃): 1.05 (t, 3H); 1.78 (m, 2H); 2.22 (s, 3H); 2.38 (t, 2H); 4.15 (br, 1H); 4.23 (s, 2H); 6.28 (m, 1H); 6.40 (m, 2H); 6.95 (br s, 1H); 7.10 (m, 1H); 7.18 (m, 2H); 7.80 (d, 1H).

### 1ib N-{4-[(4-chlorophenylamino)methyl]-2-methylphenyl}butyramide

LC/MS (m/z) 317.2 (M⁺); RT=2.72, UV purity=99.4, ELS purity=94.8 ¹H NMR (CDCl₃): 1.05 (t, 3H); 1.78 (m, 2H); 2.25 (s, 3H); 2.40 (t, 2H); 4.00 (br, 1H); 4.23 (s, 2H); 6.55 (d, 2H); 6.90 (br s, 1H); 7.10 (d, 2H); 7.18 (d, 2H); 7.78 (d, 1H).

### 1jb N-{4-[(3,4-Dichlorophenylamino)methyl]-2-methylphenyl}butyramide

LC/MS (m/z) 351.6 (M⁺); RT=3.28, UV purity=98.4, ELS purity=99.9. ¹H NMR (CDCl₃): 1.00 (t, 3H); 1.75 (m, 2H); 2.23 (s, 3H); 2.38 (t, 2H); 4.00 (br, 1H); 4.20 (s, 2H); 6.45 (d, 1H); 6.70 (s, 1H); 6.95 (br s, 1H); 7.15 (m, 3H); 7.80 (d, 1H).

### 1kb N-{4-[(4-Chloro-3-fluorophenylamino)methyl]-2-methylphenyl}butyramide

LC/MS (m/z) 334.7 (M⁺); RT=3.06, UV purity=99.7, ELS purity=99.9. ¹H NMR (CDCl₃): 1.05 (t, 3H); 1.78 (m, 2H); 2.25 (s, 3H); 2.40 (t, 2); 4.00 (br, 1H); 4.22 (s, 2H); 6.30 (m, 1H); 6.40 (m, 1H); 6.90 (br s, 1H); 7.10 (m, 1H); 7.15 (m, 2H); 7.80 (d, 1H).

### 1lb N-{2-Chloro-4-[(4-trifluoromethylphenylamino)methyl]phenyl}butyramide

LC/MS (m/z) 370.9 (M⁺); RT=3.37, WV purity=99.4, ELS purity=99.2. ¹H NMR (CDCl₃): 1.05 (t, 3H); 1.80 (m, 2H); 2.40 (t, 2H); 4.32 (s, 2H); 4.45 (br, 1H); 6.60 (d, 2H); 7.22 (d, 1H); 7.38 (s, 1H); 7.40 (d, 2H); 7.60 br s, 1H); 8.40 (d, 1H).

### 1mb N-{2-Chloro-4-[(4-fluorophenylamino)methyl]phenyl}butyramide

LC/MS (m/z) 320.9 (M⁺); RT=2.66, UV purity=94.8, ELS purity=99.2. ¹H NMR (CDCl₃): 1.05 (t, 3H); 1.80 (m, 2H); 2.40 (t, 2H); 4.10 (br, 1H); 4.22 (s, 2H); 6.55 (m, 2H); 6.85 (m, 2H); 7.22 (d, 1H); 7.40 (s, 1H); 7.60 (br s, 1H); 8.45 (d, 1H).

### 1nb N-{2-Chloro-4-[(3-fluorophenylamino)methyl]phenyl}butyramide

LC/MS (m/z) 321.0 (M⁺); RT=3.03, UV purity=99.01, ELS purity=99.8. ¹H NMR (CDCl₃): 1.05 (t, 3H); 1.80 (m, 2H); 2.43 (t, 2H); 4.15 (br 1H); 4.28 (s, 2H); 6.28 (m, 1H); 6.40 (br m, 2H); 7.08 (m, 1H); 7.22 (d, 1H); 7.38 (s, 1H); 7.55 (br s, 1); 8.48 (d, 1H).

### 1ob N-{2-Chloro-4-[(4-chlorophenylamino)methyl]phenyl}butyramide

LC/MS (m/z) 337.8 (M⁺); RT=3.11, UV purity=99.4, ELS purity=99.9. ¹H NMR (CDCl₃): 1.05 (t, 3H); 1.80 (m, 2H); 2.40 (t, 2H); 4.10 (br, 1H); 4.30 (s, 2H); 6.50 (d, 2H); 7.10 (d, 2H); 7.22 (d, 1H); 7.38 (s, 1H); 7.60 (br s, 1H); 8.40 (d, 1H).

### 1pb N-{2-Chloro-4-[(3,4-dichlorophenylamino)methyl]phenyl}butyramide

LC/MS (m/z) 371.9 (M⁺); RT=3.45, UV purity=93.5, ELS purity=95.7. ¹H NMR (CDCl₃): 1.05 (t, 3H); 1.80 (m, 2H); 2.40 (t, 2H); 4.12 (br, 1H); 4.23 (s, 2H); 6.40 (m, 1H); 6.65 (s, 1H); 7.17 (d, 1H); 7.22 (d, 1H); 7.35 (s, 1H); 7.60 (br s, 1H); 8.38 (d, 1H).

### 1qb N-{2-Chloro-4-[(4-chloro-3-fluorophenylamino)methyl]phenyl}butyramide

LC/MS (m/z) 355.24 (M⁺); RT=3.34, UV purity=98.1, ELS purity=99.5. ¹H NMR (CDCl₃): 1.05 (t, 3H); 1.80 (m, 2H); 2.42 (t, 2H); 4.25 (s, 2H); 4.35 (br, 1H); 6.35 (br m, 2H); 7.10 (m, 1H); 7.22 (d, 1H); 7.35 (s, 1H); 7.60 (br s, 1H); 8.35 (d, 1H).

### 1rb N-{2-Fluoro-4-[(3-fluorophenylamino)methyl]phenyl}butyramide

LC/MS (m/z) 305.0 (M⁺); RT=2.97, UV purity=99.4, ELS purity=99.6. ¹H NMR (CDCl₃): 1.00 (t, 3H); 1.75 (m, 2H); 2.40 (t, 2H); 4.15 (br, 1H); 4.30 (s, 2H); 6.27 (d, 1H); 6.40 (br m, 2H); 7.10 (br m, 3H); 7.30 (br s, 1H); 8.30 (m, 1H).

### 1sb N-{4-[(4-Chlorophenylamino)methyl]-2-fluorophenyl}butyramide

LC/MS (m/z) 320.0 (M⁺); RT=2.94, UV purity=99.4, ELS purity=99.9. ¹H NMR (CDCl₃): 1.00 (t, 3H); 1.80 (m, 2H); 2.40 (t, 2H); 4.15 (br, 1H); 4.30 (s, 2H); 6.50 (d, 2H); 7.10 (br m, 4H); 7.30 (br s, 1H); 8.30 (m, 1H).

### 1tb N-{2-Fluoro-4-[(4-trifluoromethylphenylamino)methyl]phenyl}butyramide

LC/MS (m/z) 354.0 (M⁺); RT=3.20, UV purity=99.6, ELS purity=99.9. ¹H NMR (CDCl₃): 1.00 (t, 3H); 1.78 (m, 2H); 2.40 (t, 2H); 4.15 (br, 1H); 4.35 (s, 2H); 6.60 (d, 2H); 7.10 (m, 2H); 7.30 (br s, 1H); 7.40 (d, 2H); 8.30 (m, 1H).

### 1ub N-{4-[(3,4-Dichlorophenylamino)methyl]-2-fluorophenyl}butyramide

LC/MS (m/z) 355.0 (M⁺); RT=3.29, UV purity=99.7, ELS purity=99.9. ¹H NMR (CDCl₃): 1.00 (t, 3H); 1.78 (m, 2H); 2.40 (t, 2H); 4.20 (br, 1H); 4.28 (s, 2H); 6.42 (d, 1H); 6.65 (s, 1H); 7.05 (m, 2H); 7.18 (d, 1H); 7.30 (br s, 1H); 8.30 (m, 1H).

### 1vb N-{4-[(4-Chloro-3-fluorophenylamino)methyl]-2-fluorophenyl}butyramide

LC/MS (m/z) 339.0 (⁺); RT=3.14, UV purity=99.4, ELS purity=97.9. ¹H NMR (CDCl₃): 1.00 (t, 3H); 1.75 (m, 2H); 2.40 (t, 2H); 4.00 (br, 1H); 4.25 (s, 2H); 6.30 (br m, 2H); 7.05 (br m, 3H); 7.30 (br s, 1H); 8.30 (m, 1H).

In Vitro and In Vivo Testing

The compounds of the invention have been tested and shown effect in one or more of the below models:

Relative Efflux Through the KCNQ2 Channel.

This exemplifies a KCNQ2 screening protocol for evaluating compounds of the present invention. The assay measures the relative efflux through the KCNQ2 channel, and was carried out according to a method described by Tang et al. (Tang, W. et. al., J. Biomol. Screen. 2001, 6, 325-331) for hERG potassium channels with the modifications described below.

An adequate number of CHO cells stably expressing voltage-gated KCNQ2 channels were plated at a density sufficient to yield a mono-confluent layer on the day of the experiment. Cells were seeded on the day before the experiment and loaded with 1 μCi/ml [⁸⁶Rb] over night. On the day of the experiment, cells were washed with a HBSS-containing buffer. Cells were pre-incubated with drug for 30 min and the ⁸⁶Rb⁺ efflux was stimulated by a submaximal concentration of 15 mM KCl in the continued presence of drug for additional 30 min. After a suitable incubation period, the supernatant was removed and counted in a liquid scintillation counter (Tricarb). Cells were lysed with 2 mM NaOH and the amount of ⁸⁶Rb⁺ was counted. The relative efflux was calculated ((CPMₛᵤₚₑᵣ/(CPMₛᵤₚₑᵣ+CPM_{cell}))_{Cmpd}/(CPMₛᵤₚₑᵣ/(CPMₛᵤₚₑᵣ+CPM_{cell}))_{15mM KCl})*100−100.

The compounds of the invention have an EC₅₀ of less than 20000 nM, in most cases less than 2000 nM and in many cases less than 200 nM. Accordingly, the compounds of the invention are considered to be useful in the treatment of diseases associated with the KCNQ family potassium channels.

Electrophysiological Patch-Clamp Recordings.

Voltage-activated KCNQ2 currents were recorded from mammalian CHO cells by use of conventional patch-clamp recordings techniques in the whole-cell patch-clamp configuration (Hamill O P et. al. Pflügers Arch 1981; 391: 85-100). CHO cells with stable expression of voltage-activated KCNQ2 channels were grown under normal cell culture conditions in CO₂ incubators and used for electrophysiological recordings 1-7 days after plating. KCNQ2 potassium channels were activated by voltage steps up to +80 mV in increments of 5-20 mV (or with a ramp protocol) from a membrane holding potential between −100 mV and −40 mV (Tatulian L et al. J Neuroscience 2001; 21 (15): 5535-5545). The electrophysiological effects induced by the compounds were evaluated on various parameters of the voltage-activated KCNQ2 current. Especially effects on the activation threshold for the current and on the maximum induced current were studied.

Some of the compounds of the invention have been tested in this test. A left-ward shift of the activation threshold or an increase in the maximum induced potassium current is expected to decrease the activity in neuronal networks and thus make the compounds useful in diseases with increased neuronal activity—like epilepsia.

Maximum Electroshock

The test was conducted in groups of male mice using corneal electrodes and administering a square wave current of 26 mA for 0.4 seconds in order to induce a convulsion characterised by a tonic hind limb extension (Wlaz et al. Epilepsy Research 1998, 30, 219-229).

Pilocarpine Induced Seizures

Pilocarpine induced seizures are induced by intraperitoneal injection of pilocarpine 250 mg/kg to groups of male mice and observing for seizure activity resulting in loss of posture within a period of 30 minutes (Starr et al. Pharmacology Biochemistry and Behavior 1993, 45, 321-325)

Electrical Seizure-Threshold Test

A modification of the up-and-down method (Kimball et al., Radiation Research 1957, 1-12) was used to determine the median threshold to induce tonic hind-limb extension in response to corneal electroshock in groups of male mice. The first mouse of each group received an electroshock at 14 mA, (0.4 s, 50 Hz) and was observed for seizure activity. If a seizure was observed the current was reduced by 1 mA for the next mouse, however, if no seizure was observed then the current was increased by 1 mA. This procedure was repeated for all 15 mice in the treatment group.

Chemical Seizure-Threshold Threshold Test

The threshold dose of pentylenetetrazole required to induce a clonic convulsion was measured by timed infusion of pentylenetetrazole (5 mg/ml at 0.5 ml/min) into a lateral tail vein of groups of male mice (Nutt et al. J Pharmacy and Pharmacology 1986, 38, 697-698).

Amygdala Kindling

Rats underwent surgery to implantation of tri-polar electrodes into the dorsolateral amygdala. After surgery the animals were allowed to recover before the groups of rats received either varying doses of test compound or the drug's vehicle. The animals were stimulated with their initial after discharge threshold +25 μA daily for 3-5 weeks and on each occasion seizure severity, seizure duration, and duration of electrical after discharge were noted. (Racine. Electroencephalography and Clinical Neurophysiology 1972, 32, 281-294).

Side Effects

Central nervous system side effects were measured by measuring the time mice would remain on rotarod apparatus (Capacio et al. Drug and Chemical Toxicology 1992, 15, 177-201); or by measuring their locomotor activity by counting the number of infra-red beams crossed in a test cage ((Watson et al. Neuropharmacology 1997, 36, 1369-1375). Hypothermic actions on the animals core body temperature of the compound were measured by either rectal probe or implanted radiotelemetry transmitters capable of measuring temperature (Keeney et al. Physiology and Behaviour 2001, 74, 177-184.

Pharmacokinetics

The pharmacokinetic properties of the compound were determined via. i.v. and p.o. dosing to Spraque Dawley rats, and, thereafter, drawing blood samples over 20 hours. Plasma concentrations were determined with LC/MS/MS.

1. A compound of formula I 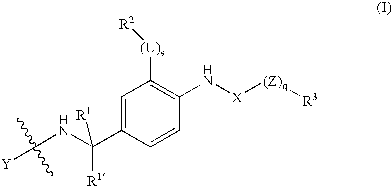
wherein
U is O, S or NR^{2′};
s is 0 or 1;
X is CO or SO₂;
Z is O, S or NR⁴, wherein R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl;
q is 0 or 1;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl;
R² is selected from the group consisting of hydrogen, halogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl, halo-C₃₋₈-cycloalk(en)yl and cyano; provided that when R² is halogen or cyano, then s is 0;
when s is 1 and U is NR^{2′} then R²′ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; or R² and R^{2′} together with the nitrogen atom to which they are attachded form a 5-8 membered saturated or unsaturated ring which optionally contains one further heteroatom;
R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; and
Y represents a group of formulae VI, VII, VIII, IX or XXX: 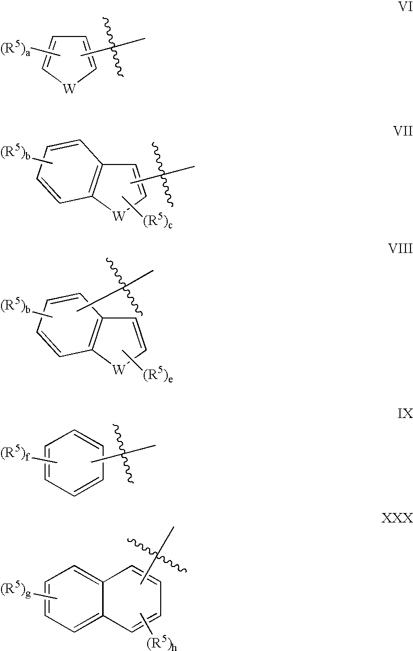
W is O or S;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3; and
each R⁵ is independently selected from the group consisting of a C₁₋₆-alk(en/yn)yl, C₃₋₈-cycloalk(en)yl, Ar, C₃₋₈-cycloalk(en)yl-C₁₋₆-alk(en/yn)yl, Ar—C₁₋₆-alk(en/yn)yl, acyl, C₁₋₆-alk(an/en/yn)yloxy, halogen, halo-C₁₋₆-alk(en/yn)yl, —CO—NR⁶R^{6′}, cyano, nitro, —NR⁷R^{7′}, —S—R⁸, —SO₂R⁸ and SO₂OR⁸, or two R⁵ substituents together with the carbon atoms to which they are attached form a 5-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; with the provisos that when R⁵ is SO₂OR⁸ then R⁸ is not —NR⁹R^{9′} and when R⁵ is SO₂R⁸, then R⁸ is not a hydrogen atom;
or salts thereof;
with the proviso that the compound of formula I is not:
N-[4-[[(4-aminophenyl)amino]methyl]phenyl]-acetamide;
N-[4-[[(4-amino-2-methylphenyl)amino]methyl]phenyl]-acetamide;
N-[4-[[(4-amino-3-methylphenyl)amino]methyl]phenyl]-acetamide;
2-[[[4-(acetylamino)phenyl]methyl]amino]-5-chloro-N-(5-chloro-2-pyridinyl)-benzamide;
N-[4-[[(3,4,5-trimethoxyphenyl)amino]methyl]phenyl]-acetamide;
N-[4-[[(5,6,7,8-tetrahydro-5,5,8,8-tetramethyl-2-naphthalenyl)amino]methyl]phenyl]-acetamide;
N-[4-[[[3-(1H-imidazol-1-ylmethyl)phenyl]amino]methyl]phenyl]-acetamide;
N-[4-[[[2-(1H-imidazol-1-ylmethyl)phenyl]amino]methyl]phenyl]-acetamide;
N-[4-[[(4-amino-3,5-dichlorophenyl)amino]methyl]phenyl]-acetamide;
N-[4-[[(2,4-diamino-6-quinazolinyl)amino]methyl]phenyl]-acetamide; or
N-[4-[[(2,4-diamino-6-quinazolinyl)amino]methyl]phenyl]-acetamide.

2. A compound according to claim 1, wherein R¹ and R^{1′} are independently selected from the group consisting of hydrogen and C₁₋₆-alk(en/yn)yl.

3. A compound according to claim 2, wherein at least one of R¹ and R^{1′} is a hydrogen atom.

4. A compound according to claim 1, wherein s is 1.

5. A compound according to claim 1, wherein s is 0.

6. A compound according to claim 1, wherein R² is selected from the group consisting of hydrogen, C₁₋₆-alk(en/yn)yl, Ar and halogen, provided that when R² is halogen, then s is 0.

7. A compound according to claim 4, wherein U is NR^{2′} and at least one of R² and R^{2′} is a hydrogen atom.

8. A compound according to claim 7, wherein both R² and R^{2′} are hydrogen atoms.

9. A compound according to claim 1, wherein X is CO.

10. A compound according to claim 1, wherein q is 0.

11. A compound according claim 1, wherein q is 1.

12. A compound according to claim 11, wherein Z is an oxygen atom.

13. A compound according to claim 1, wherein R³ is C₁₋₆-alk(en/yn)yl.

14. A compound according to claim 1, wherein Y represents a group of formulae IX or XXX.

15. A compound according to claim 1, wherein each R⁵ is independently selected from the group consisting of a C₁₋₆-alk(en/yn)yl, C₃₋₈-cycloalk(en)yl, Ar, cyano, halogen, halo-C₁₋₆-alk(en/yn)yl and C₁₋₆-alk(an/en/yn)yloxy or two adjacent R⁵ substituents together with the carbon atoms to which they are attached form a 5-8 membered ring which optionally contains one or two heteroatoms.

16. A compound selected from the group consisting of:
{2-Amino-4-[(4-tert-butylphenylamino)-methyl]-phenyl}-carbamic acid ethyl ester;
(2-Amino-4-phenylaminomethyl-phenyl)-carbamic acid ethyl ester;
[2-Amino-4-(naphthalen-2-ylaminomethyl)-phenyl]-carbamic acid ethyl ester;
[2-Amino-4-(p-tolylamino-methyl)-phenyl]-carbamic acid ethyl ester;
{2-Amino-4-[(4-trifluoromethylphenylamino)-methyl]-phenyl}-carbamic acid ethyl ester;
{2-Amino-4-[(4-chlorophenylamino)-methyl]-phenyl}-carbamic acid ethyl ester;
{2-Amino-4-[(3-fluorophenylamino)-methyl]-phenyl}-carbamic acid ethyl ester;
{2-Amino-4-[(4-fluorophenylamino)-methyl]-phenyl}-carbamic acid ethyl ester;
{2-Amino-4-[(2-fluorophenylamino)-methyl]-phenyl}-carbamic acid ethyl ester;
[2-Amino-4-(biphenyl-4-ylaminomethyl)-phenyl]-carbamic acid ethyl ester;
{2-Amino-4-[(2,4-difluorophenylamino)-methyl]-phenyl}-carbamic acid ethyl ester;
{2-Amino-4-[(4-methoxyphenylamino)-methyl]-phenyl}-carbamic acid ethyl ester;
{2-Amino-4-[(4-cyclohexylphenylamino)-methyl]-phenyl}-carbamic acid ethyl ester;
[2-Amino-4-(indan-5-ylaminomethyl)-phenyl]-carbamic acid ethyl ester;
{2-Amino-4-[(4-isopropylphenylamino)-methyl]-phenyl}-carbamic acid ethyl ester;
{2-Amino-4-[(4-butylphenylamino)-methyl]-phenyl}-carbamic acid ethyl ester;
{2-Amino-4-[(4-chloro-3-fluorophenylamino)methyl]phenyl}carbamic acid ethyl ester;
{2-Amino-4-[(2,4-dichlorophenylamino)methyl]phenyl}carbamic acid ethyl ester;
{2-Amino-4-[(2,3-dichlorophenylamino)methyl]phenyl}carbamic acid ethyl ester;
{2-Amino-4-[(3,5-dichlorophenylamino)methyl]phenyl}carbamic acid ethyl ester;
{2-Amino-4-[(3,4-dichlorophenylamino)methyl]phenyl}carbamic acid ethyl ester;
{2-Amino-4-[(3-trifluoromethylphenylamino)methyl]phenyl}carbamic acid ethyl ester;
{2-Amino-4-[(3-fluoro-4-trifluoromethylphenylamino)methyl]phenyl}carbamic acid ethyl ester;
{2-Amino-4-[(3,4-difluorophenylamino)methyl]phenyl}carbamic acid ethyl ester;
{2-Amino-4-[(4-cyanophenylamino)methyl]phenyl}carbamic acid ethyl ester;
{2-Amino-4-[(4-fluoro-3-trifluoromethylphenylamino)methyl]phenyl}carbamic acid ethyl ester;
{2-Amino-4-[(3-chloro-4-methylphenylamino)methyl]phenyl}carbamic acid ethyl ester;
{2-Amino-4-[(3-chlorophenylamino)methyl]phenyl}carbamic acid ethyl ester;
[2-Amino-4-(m-tolylaminomethyl)phenyl]carbamic acid ethyl ester;
{2-Amino-4-[1-(4-chlorophenylamino)ethyl]phenyl}carbamic acid ethyl ester;
{2-Amino-4-[1-(4-trifluoromethylphenylamino)ethyl]phenyl}carbamic acid ethyl ester;
N-{2-Amino-4-[(3-fluorophenylamino)methyl]phenyl}-2,2-dimethylpropionamide;
{4-[(4-Chlorophenylamino)methyl]phenyl}carbamic acid ethyl ester;
{4-[(4-Trifluoromethylphenylamino)methyl]phenyl}carbamic acid ethyl ester;
{4-[1-(4-Chlorophenylamino)ethyl]phenyl}carbamic acid ethyl ester;
{4-[(4-Fluorophenylamino)methyl]-2-methylphenyl}carbamic acid ethyl ester;
{4-[(4-Chlorophenylamino)methyl]-2-methylphenyl}carbamic acid ethyl ester;
{2-Methyl-4-[(4-trifluoromethylphenylamino)methyl]phenyl}carbamic acid ethyl ester;
{4-[(3,4-Difluorophenylamino)methyl]-2-methylphenyl}carbamic acid ethyl ester;
{4-[(3-Fluorophenylamino)methyl]-2-methylphenyl}carbamic acid ethyl ester;
{2-Chloro-4-[(4-chlorophenylamino)methyl]phenyl}carbamic acid ethyl ester;
{2-Chloro-4-[(4-trifluoromethyl-phenylamino)-methyl]-phenyl}-carbamic acid ethyl ester;
{2-Chloro-4-[(4-fluorophenylamino)methyl]phenyl}carbamic acid ethyl ester;
{2-Chloro-4-[(3-fluorophenylamino)methyl]phenyl}carbamic acid ethyl ester;
{2-Chloro-4-[(3,4-dichlorophenylamino)methyl]phenyl}carbamic acid ethyl ester;
{2-Chloro-4-[(4-chloro-3-fluorophenylamino)methyl]phenyl}carbamic acid ethyl ester;
{4-[(4-Chlorophenylamino)methyl]-2-fluorophenyl}carbamic acid ethyl ester;
{4-[(4-Chloro-3-fluorophenylamino)methyl]-2-fluorophenyl}carbamic acid ethyl ester;
{2-Fluoro-4-[(4-trifluoromethylphenylamino)methyl]phenyl}carbamic acid ethyl ester;
{4′-Dimethylamino-5-[(3-fluorophenylamino)methyl]biphenyl-2-yl}carbamic acid ethyl ester;
{4′-Dimethylamino-5-[(4-trifluoromethylphenylamino)methyl]biphenyl-2-yl}carbamic acid ethyl ester;
{4′-Chloro-5-[(3-fluorophenylamino)methyl]biphenyl-2-yl}carbamic acid ethyl ester;
{4′-Chloro-5-[(4-trifluoromethylphenylamino)methyl]biphenyl-2-yl}carbamic acid ethyl ester;
N-{4-[(4-chlorophenylamino)methyl]phenyl}butyramide;
N-{4-[(3,4-dichlorophenylamino)methyl]phenyl}butyramide;
N-{4-[(4-chloro-3-fluorophenylamino)methyl]phenyl}butyramide;
N-{4[(4-fluoro-phenylamino)methyl]-2-methylphenyl}butyramide;
N-{4[(3-fluorophenylamino)methyl]-2-methylphenyl}butyramide;
N-{4-[(4-chlorophenylamino)methyl]-2-methylphenyl}butyramide;
N-{4-[(3,4-dichlorophenylamino)methyl]-2-methylphenyl}butyramide;
N-{4-[(4-chloro-3-fluorophenylamino)methyl]-2-methylphenyl}butyramide;
N-{2-chloro-4-[(4-trifluoromethylphenylamino)methyl]phenyl}butyramide;
N-{2-chloro-4-[(4-fluorophenylamino)methyl]phenyl}butyramide;
N-{2-chloro-4-[(3-fluorophenylamino)methyl]phenyl}butyramide;
N-{2-chloro-4-[(4-chlorophenylamino)methyl]phenyl}butyramide;
N-{2-chloro-4-[(3,4-dichlorophenylamino)methyl]phenyl}butyramide;
N-{2-chloro-4-[(4-chloro-3-fluorophenylamino)methyl]phenyl}butyramide;
N-{2-fluoro-4-[(3-fluorophenylamino)methyl]phenyl}butyramide;
N-{4-[(4-chlorophenylamino)methyl]-2-fluorophenyl}butyramide;
N-{2-fluoro-4-[(4-trifluoromethylphenylamino)methyl]phenyl}butyramide;
N-{4-[(3,4-dichlorophenylamino)methyl]-2-fluorophenyl}butyramide; and
N-{4-[(4-chloro-3-fluorophenylamino)methyl]-2-fluorophenyl}butyramide,
or a salt thereof.

17. A pharmaceutical composition comprising a compound formula I 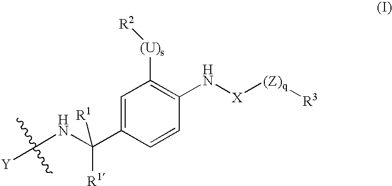
wherein
U is O, S or NR^{2′};
s is 0 or 1;
X is CO or SO₂;
Z is O, S or NR⁴, wherein R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl;
q is 0 or 1;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl;
R² is selected from the group consisting of hydrogen, halogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl, halo-C₃₋₈-cycloalk(en)yl and cyano; provided that when R² is halogen or cyano then s is 0;
when s is 1 and U is NR^{2′} then R^{2′} is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; or R² and R^{2′} together with the nitrogen atom to which they are attached form a 5-8 membered saturated or unsaturated ring which optionally contains one further heteroatom;
R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; and
Y represents a group of formulae VI, VII, VIII, IX or XXX: 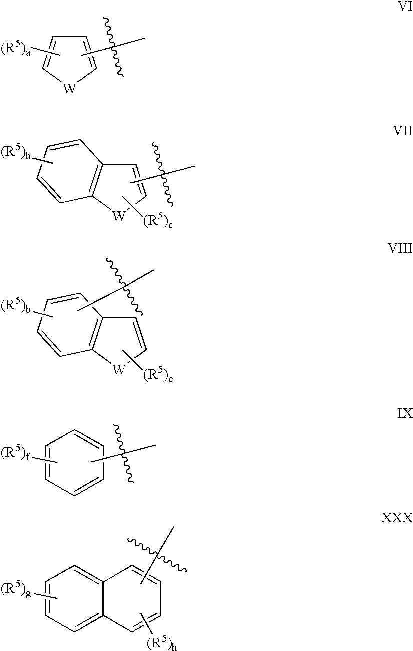
W is O or S;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3; and
each R⁵ is independently selected from the group consisting of a C₁₋₆-alk(en/yn)yl, C₃₋₈-cycloalk(en)yl, Ar, C₃₋₈-cycloalk(en)yl-C₁₋₆-alk(en/yn)yl, Ar—C₁₋₆-alk(en/yn)yl, acyl, C₁₋₆alk(an/en/yn)yloxy, halogen, halo-C₁₋₆-alk(en/yn)yl, —CO—NR⁶R^{6′}, cyano, nitro, —NR⁷R^{7′}, —S—R⁸, —SO₂R⁸ and SO₂OR⁸, or two R⁵ substituents together with the carbon atoms to which they are attached form a 5-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; with the provisos that when R⁵ is SO₂OR⁸ then R⁸ is not —NR⁹R^{9′} and when R⁵ is SO₂R⁸, then R⁸ is not a hydrogen atom;
or a pharmaceutically acceptable salt thereof,
and one or more pharmaceutically acceptable carriers or diluents.
with the proviso that the compound of formula I is not:
2-[[[4-(acetylamino)phenyl]methyl]amino]-5-chloro-N-(5-chloro-2-pyridinyl)-benzamide;
N-[4-[[(3,4,5-trimethoxyphenyl)amino]methyl]phenyl]-acetamide;
N-[4-[[(5,6,7,8-tetrahydro-5,5,8,8-tetramethyl-2-naphthalenyl)amino]methyl]phenyl]-acetamide;
N-[4-[[[3-(1H-imidazol-1-ylmethyl)phenyl]amino]methyl]phenyl]-acetamide;
N-[4-[[[2-(1H-imidazol-1-ylmethyl)phenyl]amino]methyl]phenyl]-acetamide;
N-[4-[[[4-(1H-imidazol-1-ylmethyl)phenyl]amino]methyl]phenyl]-acetamide;
N-[4-[[(4-amino-3,5-dichlorophenyl)amino]methyl]phenyl]-acetamide;
N-[4-[[(2,4-diamino-6-quinazolinyl)amino]methyl]phenyl]-acetamide; or
N-[4-[[(2,4-diamino-6-quinazolinyl)amino]methyl]phenyl]-acetamide.

18. A method of increasing ion flow in a potassium channel of a mammal, comprising administering to said mammal a compound of formula I 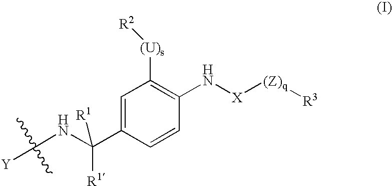
wherein
U is O, S or NR^{2′};
s is 0 or 1;
X is CO or SO₂;
Z is O, S or NR⁴, wherein R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl;
q is 0 or 1;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl;
R² is selected from the group consisting of hydrogen, halogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl, halo-C₃₋₈-cycloalk(en)yl and cyano; provided that when R² is halogen or cyano then s is 0;
when s is 1 and U is NR^{2′} then R^{2′} is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; or R² and R^{2′} together with the nitrogen atom to which they are attached form a 5-8 membered saturated or unsaturated ring which optionally contains one further heteroatom;
R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; and
Y represents a group of formulae VI, VII, VIII, IX or XXX: 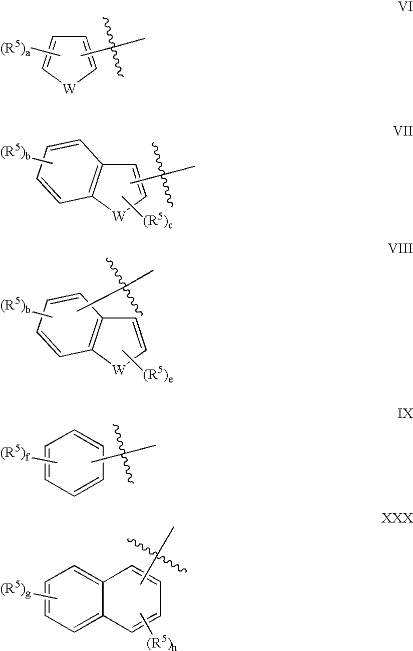
W is O or S;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3; and
each R⁵ is independently selected from the group consisting of a C₁₋₆-alk(en/yn)yl, C₃₋₈-cycloalk(en)yl, Ar, C₃₋₈-cycloalk(en)yl-C₁₋₆-alk(en/yn)yl, Ar—C₁₋₆-alk(en/yn)yl, acyl, C₁₋₆-alk(an/en/yn)yloxy, halogen, halo-C₁₋₆-alk(en/yn)yl, —CO—NR⁶R^{6′}, cyano, nitro, —NR⁷R^{7′}, —S—R⁸, —SO₂R⁸ and SO₂OR⁸, or two R⁵ substituents together with the carbon atoms to which they are attached form a 5-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; with the provisos that when R⁵ is SO₂OR⁸ then R⁸ is not —NR⁹R^{9′} and when R⁵ is SO₂R⁸, then R⁸ is not a hydrogen atom;
or salts thereof.

19. The method of claim 18 wherein administration of said compound is for the prevention, treatment or inhibition of a disorder or condition being responsive to an increased ion flow in a potassium channel.

20. The method of claim 19 wherein the disorder or condition is a seizure disorder.

21. The method of claim 19 wherein the disorder or condition is selected from the group consisting of neuropathic and migraine pain disorders.

22. The method of claim 19 wherein the disorder or condition is an anxiety disorder.

23. The method of claim 19 wherein the disorder or condition is a neurodegenerative disorder.

24. The method of claim 19 wherein the disorder or condition is a neuronal hyperexcitation state.

25. The method of claim 18 wherein the mammal is a human.

26. The method of claim 19 wherein the disorder or condition is a disorder or condition of the central nervous system.

27. The method of claim 20 wherein the seizure disorder is selected from the group consisting of convulsions, epilepsy and status epileptus.

28. The method of claim 21 wherein the neuropathic or migraine pain disorder is selected from the group consisting of allodynia, hyperalgesic pain, phantom pain, neuropathic pain related to diabetic neuropathy and neuropathic pain related to migraine.

29. The method of claim 22 wherein the anxiety disorder is selected from the group consisting of anxiety, generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia, specific phobias, anxiety disorder due to general medical condition and substance-induced anxiety disorder.

30. The method of claim 23 wherein the neurodegenerative disorder is selected from the group consisting of Alzheimer's disease, Huntington's chorea, multiple sclerosis, amyotrophic lateral sclerosis, AIDS-induced encephalopathy and other infection-related encephalopathies being caused by rubella viruses, herpes viruses, borrelia and by unknown pathogens, Creutzfeld-Jakob disease, Parkinson's disease, trauma-induced neurodegenerations.

31. The method of claim 24 wherein the neuronal hyperexcitation state is due to medicament withdrawal or intoxication.
